(19) United States
(12) Patent Application Publication
(10) Pub. No.: US 2018/0280488 A1
(43) Pub. Date: Oct. 4, 2018

(54) Methods and Compositions for Binding Complement C3 for Targeting of Immune Cells

(71) Applicant: University of Alaska Anchorage, Anchorage, AK (US)

(72) Inventor: Max Kullberg, Anchorage, AK (US)

(21) Appl. No.: 15/928,758

(22) Filed: Mar. 22, 2018

Related U.S. Application Data
(60) Provisional application No. 62/480,162, filed on Mar. 31, 2017.

(51) Int. Cl. A61K 39/00; A61K 9/127

(52) U.S. Cl. CPC A61K 39/0011; A61K 2039/57; A61K 9/127

(57) ABSTRACT

Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic. Also disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells.

### CROSS-REFERENCE TO RELATED APPLICATIONS

This application claims benefit of U.S. Provisional Application No. 62/480,162, filed Mar. 31, 2017 and is hereby incorporated herein by reference in its entirety.

### STATEMENT REGARDING FEDERALLY SPONSORED RESEARCH

This invention was made with government support under Grant P20GM103395 awarded by the National Institutes of Health/National Institute of General Medical Sciences. The government has certain rights in the invention.

### BACKGROUND

The cancer immunotherapy field has already applied considerable effort and ingenuity to improving antigen delivery to APCs. Strategies for improved antigen delivery include both in vivo and ex vivo techniques. In vivo techniques often utilize nanoparticles targeted to dendritic cells or macrophages in the body while ex vivo techniques remove dendritic cells and T cells, educate them with tumor antigens and then inject the cells back into the patient. Although ex vivo techniques can be effective, the disadvantages include the cost of individual treatment and the lack of memory T cell response because of the artificial conditions in which antigen is presented. A more practical and effective approach would be to deliver antigen in vivo where a robust and natural response can occur.

Current nanoparticle antigen delivery techniques include cationic, mannose, Fc-targeted, CD11c-targeted and CD-sign targeted liposome carriers. In addition, liposomes have been modified to be pH sensitive, fusogenic or activated by ultrasound to promote delivery of antigen to the cytoplasm of dendritic cells. All of these have had some level of success, but there are drawbacks to each of the techniques.

### BRIEF SUMMARY

Cationic nanoparticles are attractive because they bind to cell membranes which are negatively charged, but when injected systemically into the body, they aggregate and accumulate almost entirely in the lung and liver. Most of the actively targeted nanoparticles described above use antibodies or fragments of antibodies to target dendritic cells. This not only limits delivery to dendritic cells, but the challenges of scaling up and storing such complicated liposomal formulations is daunting. Fusogenic and pH sensitive liposomes aim at cytoplasmic delivery with the goal of driving a CTL response. This could be effective, but a balanced response will also require major histocompatibility (MHC) II cross presentation by dendritic cells and delivery to all three APCs. Of all the systems listed above, the mannose liposomes are the most attractive, because they use a small simple sugar to direct uptake by macrophages and dendritic cells. The advantage of the disclosed system is that the liposomes are targeted via complement C3 to the complement receptor which has been shown to be a potent APC activator. Targeting antigen to all three APCs should allow for a more effective and balanced immune response.

Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic.

Disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells

In some instances of the disclosed methods, the therapeutic can be an antigen of interest, an activating compound, or a therapeutic compound. In some instances, the antigen of interest can be a cancer antigen.

In some instances of the disclosed methods further comprise coating the nanoparticle with activated C3 prior to administering the nanoparticle to the subject. In some instances, the activated C3 can be isolated from the subject prior to coating the nanoparticle. In some instances, the activated C3 coated on the nanoparticle is synthetic.

In some instances of the disclosed methods, the antigen presenting cells can be macrophages, dendritic cells or B cells. In some instances, the antigen presenting cells comprise C3 receptor.

In some instances of the disclosed methods, the method further comprises administering a second therapeutic to the subject. In some instances, the second therapeutic can be a known therapeutic for the disease or condition being treated. In some instances, the nanoparticle comprises both the therapeutic and the second therapeutic. In some instances, the therapeutic and the second therapeutic are administered separately.

In some instances of the disclosed methods, the nanoparticle can be a liposome.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows OPSS-liposomes specifically bind complement C3 proteins. OPSS-liposomes and control-liposomes (control) were exposed to serum containing complement C3. (A) Colloidal gold stain shows all serum proteins bound to liposomes. (B) Immunodetection with anti-C3 antibody shows that OPSS-liposomes bind complement C3 and its activated fragments, while control-liposomes do not. The migration of molecular weight markers is indicated on the left side of the blot.

FIG. 2 shows OPSS-liposomes are internalized by white blood cells with complement receptor 3 (CR3). Uptake of liposomes was detected by the presence of rhodamine in the targeted cells. Binding of complement C3 to OPSS-liposomes directs internalization into cells via CR3 (CD11b). Non-OPSS-liposomes (control) display limited internalization by cells. OPSS-liposomes and control-liposomes are not readily internalized into cells when incubated in serum depleted of complement C3 (C3−), demonstrating the necessity of the C3-OPSS complex for internalization.

FIG. 3 shows flow cytometry gating strategy for isolating myeloid derived suppressor cells (MDSC). Leukocytes were selected as CD45+. Monocytic myeloid derived suppressor cells (M-MDSC) were isolated based on the phenotype: CD33+/Hi, CD11b+, HLA-DRLow/neg, CD14Hi. Isolation of granulocytic myeloid derived suppressor cells (G-MDSC) was based on: CD33+/Low, CD11b+, HLA-DRneg, CD14neg, CD15+.

FIG. 4 shows myeloid derived suppressor cells (MDSC) internalize OPSS-liposomes. M-MDSC show high uptake of rhodamine labeled OPSS-liposomes. G-MDSC also display high uptake of rhodamine labeled OPSS-liposomes. In contrast, both M-MDSC and G-MDSC showed limited uptake of control-liposomes or OPSS-liposomes incubated in serum depleted of complement C3 (C3−). Data are expressed as mean±SE (n=5).

FIG. 5 shows flow cytometry gating strategy for isolating antigen presenting cells (APC). B cells were isolated based on SSC vs. FSC and on the phenotype: HLA-DR+, CD20+. Macrophages were HLA-DR+, CD14+/Hi. Myeloid dendritic cells were HLA-DR+, CD14Low/neg, CD11c+.

FIG. 6 shows antigen presenting cells (APCs) take up OPSS-liposomes. Both OPSS-liposomes and control liposomes are rhodamine-tagged. Rhodamine labeled OPSS-liposomes incubated in serum with complement C3 are internalized by APCs, whereas non-OPSS-liposomes (control) in C3+ serum are not internalized by cells. OPSS-liposomes and control-liposomes incubated in serum depleted of complement C3 (C3−) are not internalized by cells. Data are expressed as mean±SE (n=5).

FIG. 7 shows lymphocyte internalization of liposomes. All liposomes are rhodamine-labeled in order to detect uptake by cells. Rhodamine-labeled OPSS-liposomes are not internalized readily by T cells or Natural Killer cells. B cells show a high degree of internalization of C3-bound OPSS-liposomes, but limited internalization of control-liposomes or OPSS-liposomes incubated in serum depleted of complement C3 (C3−). Data are expressed as mean±SE (n=5)

FIGS. 8A and 8B show in vivo biodistribution of C3− and control-liposomes. C3-liposomes administered systemically to tumor-bearing mice are found in CD11b+ (CR3) cells present in the blood, tumor and spleen (A). Fluorescent microscopy of blood and tissue slices shows that G-MDSC that engulf C3-liposomes are stained heavily with rhodamine liposomes while control liposomes show limited presence in these tissues (B).

FIG. 9 shows Ova C3-liposomes target antigen to APC, stimulating T cells. C3-liposomes that encapsulate ovalbumin were incubated with bone marrow derived dendritic cells for 24 h. T cells that are reactive to ovalbumin and express GFP when activated were then added for 24 h to the dendritic cells. Fluorescent microscopy shows that Ova C3-liposomes deliver antigen and activate T cells more readily than the same concentration of non-encapsulated ovalbumin or PBS. The concentration of non-encapsulated ovalbumin had to be increased 7000-fold to achieve similar T-cell stimulation, showing the potency of Ova C3-liposomes. Flow cytometry quantitates the increase in GFP expression, associated with activated T cells.

FIG. 10 shows C3-liposomes activate dendritic cells. Bone marrow derived dendritic cells that have been incubated for 48 h with C3-liposomes show increased levels of activation markers (CD80, CD83 and CD86) compared to cells incubated with PBS. A fourth activation marker, CD40 was not found in a higher percentage of cells, but appears to show increased expression on the surface of dendritic cells. This activation of dendritic cells is necessary to avoid T-cell tolerance and tumor immunosuppression.

FIGS. 11A, 11B, and 11C show C3-liposomes improve antigen delivery to monocytic APCs. Rhodamine-labeled OPSS-liposomes containing DQ-OVA incubated in human serum containing complement C3 (C3-liposomes) are taken up by (a) macrophages, (b) dendritic cells, and (c) B cells. Rhodamine-labeled control-liposomes and OPSS-liposomes lacking complement C3 are not taken up by cells. When compared with an equal amount of non-encapsulated DQ-OVA (Free DQ-OVA), C3-liposomes improve uptake and processing of DQ-OVA in macrophages and dendritic cells. B cells internalize C3-liposomes, but do not process DQ-OVA. Data are expressed as mean±standard error (n=3).

FIG. 12 shows C3-liposomes increase delivery and processing of DQ-OVA. Human monocytes were incubated with rhodamine-labeled C3-liposomes containing DQ-OVA for 3 hours. Monocytes were rinsed and imaged for rhodamine-labeled liposomes and for DQ-OVA, which fluoresces as FITC when processed for presentation. DQ-OVA C3-liposomes show high uptake into monocytes (rhodamine) and improve delivery of DQ-OVA when compared to non-encapsulated DQ-OVA at the same concentration (DQ-OVA Free).

FIGS. 13A and 13B show C3-liposomes targeting BMDCs increase T cell activation. (A) Fluorescence microscopy. (B) Graph representing microscopy data. Bone marrow-derived dendritic cells were incubated with rhodamine-labeled C3-liposomes containing OVA for 24 hours. Dendritic cells were rinsed, and T cells engineered to express GFP when presented with OVA epitopes were co-cultured with the dendritic cells for 24 hours. C3-liposome treatment resulted in increased T cell activation, compared to non-OPSS liposomes (control-liposomes) containing OVA and an equivalent amount of non-encapsulated OVA (free OVA). Data are expressed as mean±standard error (n=3).

FIGS. 14A and 14B. OVA C3-liposomes result in reduced tumor volume of both treated and distal established tumors. Mice were injected with A20-OVA cells on both flanks and treated once tumors became palpable (approximately 10-14 days). Intra-tumor injections of the 4 treatment groups were given consecutively on days 1 & 2, and then every other day for a total of 7 injections. Tumor measurements were made before all injections. (a) Injected tumor volume and (b) Non-injected distal tumor volume (mm3). Data are expressed as mean±standard error (n=3).

FIGS. 15A and 15B show OVA C3-liposome treatments result in a lower percentage of MDSCs (A) and a higher percentage of B cells (B). Mice from tumor volume experiments were euthanized and peripheral blood collected. Percentages of MDSCs (CD11b+, Ly6C high) and B cells (CD20+) were analyzed by flow cytometry. Cell populations are displayed as percentages of the total number of white blood cells. Data are expressed as mean±standard error (n=3).

FIG. 16 shows OVA treatments result in higher circulating levels of anti-OVA IgG1. Mice from tumor volume experiments were euthanized and plasma collected. Anti-OVA IgG1 levels were analyzed via ELISA. Data are expressed as mean±standard error (n=3).

FIG. 17 is a graph showing C3-liposomes activate monocytes even without CpG. Each set of bars from left to right shows PBS, CpG C3-liposomes, and Empty C3-liposomes.

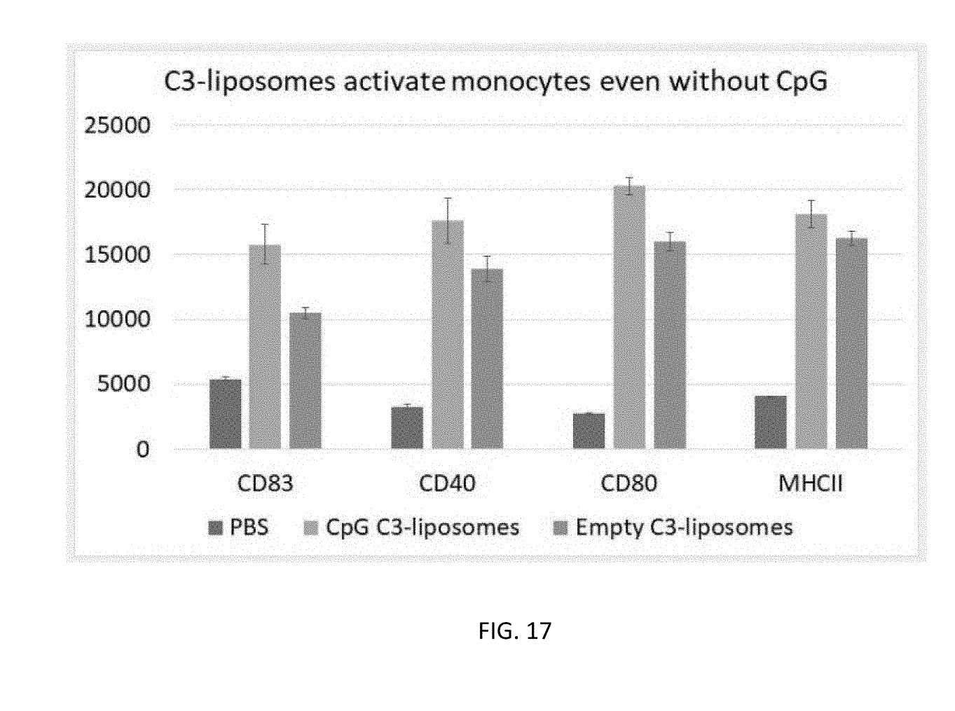

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nanoparticle is disclosed and discussed and a number of modifications that can be made to a number of molecules including the nanoparticle are discussed, each and every combination and permutation and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an antigen presenting cell” includes a plurality of such antigen presenting cells, reference to “the nanoparticle” is a reference to one or more nanoparticles and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

The term “therapeutic” refers to a treatment, therapy, or drug that can treat a disease or condition or that can ameliorate one or more symptoms associated with a disease or condition. As used herein, a therapeutic can refer to an antigen of interest, an activating compound, or a therapeutic compound, including, but not limited to proteins, peptides, nucleic acids (e.g. CpG oligonucleotides), small molecules, vaccines, allergenic extracts, antibodies, gene therapies, other biologics or small molecules.

As used herein, the term “subject” or “patient” refers to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the term “treating” refers to partially or completely alleviating, ameliorating, relieving, preventing, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “targets” refers to a mechanism in which nanoparticle-C3 conjugates find a specific cell type (e.g. antigen presenting cells) and bind to, interact with, or form a complex with the specific cell type. For example, a nanoparticle-C3 conjugate can target an antigen presenting cell, wherein the C3 from the nanoparticle-C3 conjugate binds to a complement receptor on the antigen presenting cell. The interaction or binding of C3 with a complement receptor is well known in the art.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Treating

Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic. As used throughout, antigen presenting cells being “exposed to” a therapeutic can include the transporting of a therapeutic into the antigen presenting cell. In some aspects, being “exposed to” can include binding to a receptor on the surface of the antigen presenting cell and either remaining on the surface or being internalized. The action of the therapeutic can take place on the surface or interior of the cells, including endosomal compartments, cytoplasm and nucleus.

In some instances, the disease can be cancer. In some instances, the disease or condition can be any disease or condition in which triggering or activating antigen presenting cells to present a desired antigen on their surface would be beneficial. For example, the disease or condition can be, but is not limited to, an infection (e.g. bacterial or viral), an autoimmune disease (e.g. lupus, rheumatoid arthritis, multiple sclerosis, hashimoto's, etc.), toxicity or cancer. Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic, wherein the method further comprises coating the nanoparticle with activated C3 prior to administering the nanoparticle to the subject. In some aspects, the activated C3 prior is obtained from the subject being treated. In some instances, nanoparticles can be coated with C3 and then later activated either in vitro or in vivo.

Disclosed are methods of treating a disease or condition in a subject comprising administering to a subject a nanoparticle, wherein the nanoparticle comprises a therapeutic and activated C3 coated on the surface of the nanoparticle, wherein the nanoparticle targets antigen presenting cells, wherein the antigen presenting cells are then exposed to the therapeutic. In some instances, the method can further comprise isolating the activated C3 from the subject prior to coating the nanoparticle. In some instances, the method can further comprise isolating the activated C3 from the subject and coating the nanoparticle with the subject's own activated C3 prior to administering the nanoparticle to the subject. In some instances, serum containing activated C3 can be obtained prior to coating the nanoparticle with activated C3. The serum can be from the subject being treated or from another subject. In other words, the serum can be native or non-native to the subject being treated. Thus, in some instances, the method can further comprise obtaining serum containing activated C3 and incubating the serum with the nanoparticle prior to administering the nanoparticle to the subject, wherein incubating the serum with the nanoparticle allows for the activated C3 in the serum to bind to or coat the nanoparticle. In some instances, synthetic activated C3 can be used to coat a nanoparticle prior to administering the nanoparticle to the subject. As such, in some instances the activated C3 can be native to the subject or non-native to the subject.

Disclosed are methods of treating a disease or condition in a subject comprising coating a nanoparticle with activated C3 forming a nanoparticle-C3 conjugate, administering to a subject a nanoparticle-C3 conjugate, wherein the nanoparticle-C3 conjugate comprises a therapeutic, wherein the nanoparticle-C3 conjugate targets antigen presenting cells, wherein the antigen presenting cells are then exposed to the therapeutic.

Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic further comprising administering a second therapeutic to the subject. In some instances, the second therapeutic can be a known therapeutic for the disease or condition being treated. For example, if the disease being treated is cancer, the second therapeutic can be any of a wide variety of known cancer therapeutics such as but not limited to, chemotherapy, radiation, and any of the known cancer drugs.

In some instances, the nanoparticle comprises both the therapeutic and the second therapeutic. Therefore, the therapeutic and the second therapeutic can be administered simultaneously. Administering simultaneously can include administering the therapeutic in the same formulation as the nanoparticle or in separate formulations. In some instances, the therapeutic and the second therapeutic are administered separately. Administering separately can include administering at the same time but in different formulations or can mean administering at different times. In some instances, administering at different times can be administering the therapeutic and the second therapeutic within 15, 30, 45, or 60 minutes of each other. In some instances, administering at different times can be administering the therapeutic and the second therapeutic within 1, 2, 3, 4, 5, 6, 7, 8, 9, 10, 11, 12, 13, 14, 15, 16, 17, 18, 19, 20, 21, 22, 23, or 24 hours of each other. In some instances, administering at different times can be administering the therapeutic and the second therapeutic within 1, 2, 3, 4, 5, 6, or 7 days of each other. In some instances, administering at different times can be administering the therapeutic and the second therapeutic within 1, 2, 3, 4, 5, 6, 7, 8, 9, 10, 11, or 12 months of each other.

Disclosed are methods of treating a disease or condition in a subject comprising administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic, wherein the therapeutic can be an antigen of interest, an activating compound, or a therapeutic compound. In some instances, the therapeutic can be, but is not limited to, proteins, peptides, nucleic acids, small molecules and other biologics.

In some instances, the antigen of interest can be a cancer antigen. For example, a cancer antigen can include, but is not limited to, Melanoma Associated Antigen (MAGE), Epithelial Tumor Antigen (ETA), Human Epidermal Growth Factor Receptor 2 (Her-2), CA-125, Carcinoembryonic antigen or abnormal mutations of P53 and RAS. In some instances, the antigen of interest can be any antigen involved in the disease or condition process of the disease or condition being treated. In some instances, the antigen of interest acts as part of a vaccine. For example, the antigen of interest can help prevent bacterial or viral infections or the development of cancer. Thus, in some instances, the disclosed methods of treating can be methods of vaccinating.

In some instances, a therapeutic compound can be any compound known to treat the disease or condition being treated. For example, if the disease being treated is cancer, the second therapeutic can be any of a wide variety of known cancer therapeutics such as but not limited to, cisplatin, Gleevec, Gemcitabine, Methotrexate, Trastuzumab. In some instances, a therapeutic compound can be a chemical compound, a protein, or a nucleic acid.

In some instances, an activating compound can be, but is not limited to, agonists of toll-like receptors (TLR) including but not limited to CpG oligonucleotide repeats, Polyinosinic:polycytidylic acid (Poly IC), lipopolysachrides (LPS) and drugs that stimulate TLR.

Also disclosed are methods of treating a disease or condition in a subject comprising administering to a subject a nanoparticle, wherein upon administration the nanoparticle binds to activated C3 present in the subject forming a nanoparticle-C3 conjugate, wherein the nanoparticle-C3 conjugate targets antigen presenting cells, wherein the antigen presenting cells are activated upon exposure to the nanoparticle-C3 conjugate.

Also disclosed are methods of treating a disease or condition in a subject comprising coating a nanoparticle with C3 forming a nanoparticle-C3 conjugate, administering to a subject the nanoparticle-C3 conjugate, wherein the nanoparticle-C3 conjugate targets antigen presenting cells, wherein the antigen presenting cells are activated upon exposure to the nanoparticle-C3 conjugate. In some instances, the C3 coated on the nanoparticle is activated. In some instances, the C3 coated on the nanoparticle is activated after the nanoparticle-C3 conjugate is formed. The activation can occur in vitro or in vivo. In some instances, the C3 coated on the nanoparticle can be obtained from the subject being treated or from a different subject. In some instances, the method can further comprise isolating activated C3 from the subject and coating the nanoparticle with the subject's own activated C3 prior to administering the nanoparticle to the subject.

In some instances, the antigen presenting cells can be macrophages, dendritic cells or B cells. In some instances, antigen presenting cells can be any cell known to be capable of presenting or displaying antigen on its surface via a major histocompatibility complex.

In some instances, the antigen presenting cells comprise at least one C3 receptor. In some instances, C3 receptor is upregulated prior to administration of a nanoparticle in order to increase expression levels of C3 receptor on the surface of antigen presenting cells. In some instances, the upregulation of the C3 receptor can be induced prior to administration of a nanoparticle.

### C. Methods of Delivering

Disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells.

Disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells further comprising coating the nanoparticle with activated C3 prior to administering the nanoparticle to the subject. In some instances, the method can further comprise isolating the activated C3 from the subject prior to coating the nanoparticle. In some instances, the method can further comprise isolating the activated C3 from the subject and coating the nanoparticle with the subject's own activated C3 prior to administering the nanoparticle to the subject. In some instances, serum containing activated C3 can be obtained prior to coating the nanoparticle with activated C3. The serum can be from the subject being treated or from another subject. In other words, the serum can be native or non-native to the subject being treated. Thus, in some instances, the method can further comprise obtaining serum containing activated C3 and incubating the serum with the nanoparticle prior to administering the nanoparticle to the subject, wherein incubating the serum with the nanoparticle allows for the activated C3 in the serum to bind to or coat the nanoparticle. In some instances, synthetic activated C3 can be used to coat a nanoparticle prior to administering the nanoparticle to the subject. As such, in some instances, the activated C3 can be native to the subject or non-native to the subject.

Disclosed are methods of delivering a therapeutic to antigen presenting cells comprising coating a nanoparticle with activated C3 forming a nanoparticle-C3 conjugate, administering to a subject a nanoparticle-C3 conjugate, wherein the nanoparticle-C3 conjugate comprises a therapeutic, wherein upon administration the nanoparticle binds to activated C3 present in the subject forming a nanoparticle-C3 conjugate, wherein the nanoparticle-C3 conjugate targets antigen presenting cells.

Disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells, further comprising administering a second therapeutic to the subject.

In some instances, the nanoparticle comprises both the therapeutic and the second therapeutic. Therefore, the therapeutic and the second therapeutic can be administered simultaneously. In some instances, the therapeutic and the second therapeutic are administered separately. Administering separately can include administering at the same time but in different formulations or can mean administering at different times. In some instances, administering at different times can be administering the therapeutic and the second therapeutic within 15, 30, 45, or 60 minutes of each other. In some instances, administering at different times can be administering the therapeutic and the second therapeutic within 1, 2, 3, 4, 5, 6, 7, 8, 9, 10, 11, 12, 13, 14, 15, 16, 17, 18, 19, 20, 21, 22, 23, or 24 hours of each other. In some instances, administering at different times can be administering the therapeutic and the second therapeutic within 1, 2, 3, 4, 5, 6, or 7 days of each other. In some instances, administering at different times can be administering the therapeutic and the second therapeutic within 1, 2, 3, 4, 5, 6, 7, 8, 9, 10, 11, or 12 months of each other.

Disclosed are methods of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells, wherein the therapeutic can be an antigen of interest, an activating compound, or a therapeutic compound. In some instances, the therapeutic can be, but is not limited to, proteins, peptides, nucleic acids, small molecules and other biologics.

In some instances, the antigen of interest can be a cancer antigen. In some instances, the antigen of interest can be any antigen involved in the disease or condition process of the disease or condition being treated.

In some instances, a therapeutic compound can be any compound known to treat the disease or condition being treated. For example, if the disease being treated is cancer, the second therapeutic can be any of a wide variety of known cancer therapeutics such as but not limited to, cisplatin, Gleevec, Gemcitabine, Methotrexate, Trastuzumab. In some instances, a therapeutic compound can be a chemical compound, a protein, or a nucleic acid.

In some instances, an activating compound can be, but is not limited to, agonists of toll-like receptors (TLR) including but not limited to CpG oligonucleotide repeats, Polyinosinic:polycytidylic acid (Poly IC), lipopolysachrides (LPS) and drugs that stimulate TLR.

Also disclosed are methods of delivering nanoparticle-C3 conjugates to antigen presenting cells comprising administering to a subject a nanoparticle, wherein upon administration the nanoparticle binds to activated C3 present in the subject forming a nanoparticle-C3 conjugate, wherein the nanoparticle-C3 conjugate targets antigen presenting cells. In some instances, the antigen presenting cells are activated upon exposure to the nanoparticle-C3 conjugate.

Disclosed are methods of delivering nanoparticle-C3 conjugates to antigen presenting cells comprising coating a nanoparticle with C3 forming a nanoparticle-C3 conjugate, administering to a subject the nanoparticle-C3 conjugate, wherein the nanoparticle-C3 conjugate targets antigen presenting cells. In some instances, the antigen presenting cells are activated upon exposure to the nanoparticle-C3 conjugate. In some instances, the C3 coated on the nanoparticle is activated. In some instances, the C3 coated on the nanoparticle is activated after the nanoparticle-C3 conjugate is formed. The activation can occur in vitro or in vivo. In some instances, the C3 coated on the nanoparticle can be obtained from the subject being treated or from a different subject. In some instances, the method can further comprise isolating activated C3 from the subject and coating the nanoparticle with the subject's own activated C3 prior to administering the nanoparticle to the subject.

In some instances, the antigen presenting cells can be macrophages, dendritic cells or B cells. In some instances, antigen presenting cells can be any cell known to be capable of presenting or displaying antigen on its surface via a major histocompatibility complex.

In some instances, the antigen presenting cells comprise at least one C3 receptor. In some instances, C3 receptor is upregulated prior to administration of a nanoparticle in order to increase expression levels of C3 receptor on the surface of antigen presenting cells. In some instances, the upregulation of the C3 receptor can be induced prior to administration of a nanoparticle.

In some aspects, the nanoparticle is a liposome.

### D. Methods of Reducing Tumor Growth

Disclosed are methods of reducing tumor growth in a subject comprising administering to a subject a nanoparticle, wherein the nanoparticle comprises a tumor antigen, wherein upon administration the nanoparticle binds to activated C3 present in the subject forming a nanoparticle-C3 conjugate, wherein the nanoparticle-C3 conjugate targets antigen presenting cells, wherein the antigen presenting cells present the tumor antigen to T cells, wherein the T cells become activated and target tumors expressing the tumor antigen.

Disclosed are methods of reducing tumor growth in a subject comprising administering to a subject a nanoparticle pre-coated with C3 forming a nanoparticle-C3 conjugate, wherein the nanoparticle comprises a tumor antigen, wherein the nanoparticle-C3 conjugate targets antigen presenting cells, wherein the antigen presenting cells present the tumor antigen to T cells, wherein the T cells become activated and target tumors expressing the tumor antigen. In some instances, the C3 pre-coated on the nanoparticle is activated. In some instances, the nanoparticles can be pre-coated with C3 and then later activated either in vitro or in vivo. In some instances, the method can further comprise isolating the activated C3 from the subject prior to coating the nanoparticle. In some instances, the method can further comprise isolating the activated C3 from the subject and coating the nanoparticle with the subject's own activated C3 prior to administering the nanoparticle to the subject. In some instances, serum containing activated C3 can be obtained prior to coating the nanoparticle with activated C3. The serum can be from the subject being treated or from another subject. In other words, the serum can be native or non-native to the subject being treated. Thus, in some instances, the method can further comprise obtaining serum containing activated C3 and incubating the serum with the nanoparticle prior to administering the nanoparticle to the subject, wherein incubating the serum with the nanoparticle allows for the activated C3 in the serum to bind to or coat the nanoparticle. In some instances, synthetic activated C3 can be used to coat a nanoparticle prior to administering the nanoparticle to the subject. As such, in some instances the activated C3 can be native to the subject or non-native to the subject.

In some instances, the antigen presenting cells can be macrophages, dendritic cells or B cells. In some instances, antigen presenting cells can be any cell known to be capable of presenting or displaying antigen on its surface via a major histocompatibility complex.

In some instances, the antigen presenting cells comprise at least one C3 receptor. In some instances, C3 receptor is upregulated prior to administration of a nanoparticle in order to increase expression levels of C3 receptor on the surface of antigen presenting cells. In some instances, the upregulation of the C3 receptor can be induced prior to administration of a nanoparticle.

Disclosed are combination treatments wherein any of the disclosed methods of reducing tumor growth further comprising administering a therapeutic to the subject. Thus, the tumors can be attacked by the activated T cells that are tumor antigen specific and the therapeutic can perform its therapeutic effect. The therapeutic can be a known therapeutic for treating tumors, such as but not limited to, chemotherapy, radiation, and any of the known cancer drugs. In some instances, the nanoparticle comprises both the therapeutic and the second therapeutic. In some instances, the nanoparticle and the therapeutic can be administered simultaneously. Administering simultaneously can include administering the therapeutic in the same formulation as the nanoparticle or in separate formulations. In some instances, the nanoparticle and the therapeutic are administered separately. Administering separately can include administering at the same time but in different formulations or can mean administering at different times. In some instances, administering at different times can be administering the nanoparticle and the therapeutic within 15, 30, 45, or 60 minutes of each other. In some instances, administering at different times can be administering the nanoparticle and the therapeutic within 1, 2, 3, 4, 5, 6, 7, 8, 9, 10, 11, 12, 13, 14, 15, 16, 17, 18, 19, 20, 21, 22, 23, or 24 hours of each other. In some instances, administering at different times can be administering the nanoparticle and the therapeutic within 1, 2, 3, 4, 5, 6, or 7 days of each other. In some instances, administering at different times can be administering the nanoparticle and the therapeutic within 1, 2, 3, 4, 5, 6, 7, 8, 9, 10, 11, or 12 months of each other.

A tumor antigen can be an antigen expressed by a tumor. In some instances, the tumor antigen is either tumor specific or is overexpressed in tumors compared to healthy tissue.

Disclosed are methods of reducing tumor growth in a subject comprising administering to a subject a nanoparticle, wherein upon administration the nanoparticle binds to activated C3 present in the subject forming a nanoparticle-C3 conjugate, wherein the nanoparticle-C3 conjugate targets antigen presenting cells, wherein the antigen presenting cells are activated upon exposure to the nanoparticle-C3 conjugate, wherein the activated antigen presenting cells present a tumor antigen to T cells, wherein the T cells become activated and target tumors expressing the tumor antigen. The presence of the nanoparticle-C3 conjugate can help activate or trigger the immune system against tumors. For example, the ability of the nanoparticle-C3 conjugate to activate antigen presenting cells can result in the activated antigen presenting cells now presenting tumor antigen to T cells which can target tumors.

Disclosed are methods of reducing tumor growth in a subject comprising administering to a subject a nanoparticle pre-coated with C3 forming a nanoparticle-C3 conjugate, wherein the nanoparticle-C3 conjugate targets antigen presenting cells, wherein the antigen presenting cells are activated upon exposure to the nanoparticle-C3 conjugate, wherein the activated antigen presenting cells present a tumor antigen to T cells, wherein the T cells become activated and target tumors expressing the tumor antigen. In some instances, the nanoparticles can be pre-coated with C3 and then later activated either in vitro or in vivo. In some instances, the method can further comprise isolating the activated C3 from the subject prior to coating the nanoparticle. In some instances, the method can further comprise isolating the activated C3 from the subject and coating the nanoparticle with the subject's own activated C3 prior to administering the nanoparticle to the subject. In some instances, serum containing activated C3 can be obtained prior to coating the nanoparticle with activated C3. The serum can be from the subject being treated or from another subject. In other words, the serum can be native or non-native to the subject being treated. Thus, in some instances, the method can further comprise obtaining serum containing activated C3 and incubating the serum with the nanoparticle prior to administering the nanoparticle to the subject, wherein incubating the serum with the nanoparticle allows for the activated C3 in the serum to bind to or coat the nanoparticle. In some instances, synthetic activated C3 can be used to coat a nanoparticle prior to administering the nanoparticle to the subject. As such, in some instances the activated C3 can be native to the subject or non-native to the subject.

### E. Nanoparticles

In all of the disclosed methods of treating, delivery, and reducing tumor growth, the nanoparticle contains lipids on the outer surface. For example, in some instances, the nanoparticle can be a liposome.

In some instances, nanoparticles are not lipid based but rather contain a group that binds to activated C3. In some instances the nanoparticle can contain a group that binds to C3 which is then later activated. Non-lipid nanoparticles can be comprised on dendrimers, polymers, or synthetic materials such as silicon.

In some instances, the lipids on the outer surface of the nanoparticles form a lipid bilayer. In some instances, the lipids on the outer surface of the nanoparticles form a single layer of lipids.

In some instances, the disclosed nanoparticles can form a bond with an exposed sulfhydryl group on the activated C3. Thus, in some instances, the disclosed nanoparticles are coated with a subject's own C3 or synthetic C3.

In some instances, the liposomes can be cationic liposomes (e.g., DOTMA, DOPE, DC-cholesterol) or anionic liposomes. Liposomes can further comprise targeting moieties to facilitate targeting the liposome to a particular cell, if desired. Administration of a cationic liposome can be administered to the blood, to a target organ, or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355.

In the methods described herein, delivery of the nanoparticles to cells can be via a variety of mechanisms. As one example, delivery can be via a liposome, using commercially available liposome preparations such as LIPOFECTIN™, LIPOFECTAMINE (GIBCO-BRL, Gaithersburg, Md.), SUPERFECT (Qiagen, Hilden, Germany) and TRANSFECTAM (Promega Biotec, Madison, Wis.), as well as other liposomes developed according to procedures standard in the art.

### F. Administering

In the methods described herein, administration or delivery of the nanoparticle to a subject can be via a variety of mechanisms. For example, the nanoparticle can be formulated as a pharmaceutical composition.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

As described herein, the nanoparticles or therapeutics can be administered in a pharmaceutically acceptable carrier and can be delivered to the subject's cells in vivo or ex vivo by a variety of mechanisms well-known in the art (e.g., liposome fusion, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for delivering a therapeutic to antigen presenting cells, the kit comprising a nanoparticle and at least one therapeutic. The kits also can contain lipids or activated C3.

### EXAMPLES

In one aspect, the liposomal system disclosed herein combines practical simplicity with efficient delivery to all three types of antigen presenting cells (APCs). The advantage of the disclosed system is that the liposomes are targeted via complement C3 to the complement receptor which has been shown to be a potent APC activator. In addition, the C3-liposomes are the first system that can deliver to not only macrophages and dendritic cells, but to B cells as well. B cell antigen presentation is critical for CD4 T cell activation and may even be involved in cross presentation to CTLs11,12. Targeting antigen to all three APCs should allow for a more effective and balanced immune response.

In this example, liposomes were used that contain a lipid conjugated to a small disulfide forming group. When injected into a mouse, the liposomes form a disulfide bond with activated C3 which displays an exposed sulfhydryl group. This binding was shown to be efficient and specific. With complement C3 displayed on their surface, liposomes are engulfed by all cells that display receptors for C3. Using human blood in vitro, selective and high level of uptake has been shown into all three APCs, neutrophils and myeloid derived suppressor cells (MDSCs). Unlike targeting systems that require ligand or antibody conjugation, this drug delivery system does not require complex chemistry and could be efficiently increased to pharmaceutically relevant quantities. The liposomes were shown to target the receptors for activated C3 present in both mice and humans, allowing for a smooth transition from animal experiments to human experiments. Finally, since the system can use the patient's own C3 protein, the liposomes should not display the immunogenicity and toxicity associated with injection of foreign antibodies and targeting ligands. By delivering to all three APCs through complement driven internalization, this system has the potential to improve on currently available techniques for tumor antigen presentation.

A. Complement C3 Dependent Uptake of Targeted Liposomes into Human Macrophages, B Cells, Dendritic Cells, Neutrophils and MDSCs

1. Introduction

Dendritic cells, B cells, macrophages, neutrophils and myeloid derived suppressor cells (MDSCs) are all involved in regulation of the immune response against cancer. The first step in an adaptive immune response against a tumor is carried out by antigen presenting cells (APCs), which include the dendritic cells, B cells and macrophages. After engulfing tumor cells, endocytic processing in APCs results in antigen presentation by major histocompatibility complexes to T helper and cytotoxic T cells. Opposing this immunostimulatory action are immunosuppressive cells. The tumor microenvironment recruits and promotes the production of numerous suppressive cell types, including pro-tumor M2 macrophages, N2 neutrophils, and MDSCs, which produce suppressive cytokines such as IL-10 and TGF-β, reactive oxygen species (ROS), nitric oxide synthetase and arginase to inhibit cytotoxic T cells. Whether targeting antigen to APCs or delivering drugs to relieve immunosuppression, the cancer immunotherapy field would benefit from a nanoparticle delivery system to both cell types. A system that targets the receptor for complement C3 has been developed, which is a commonality among dendritic cells, B cells, macrophages, neutrophils and MDSCs.

Various strategies have been employed by the nanoparticle field to target macrophages, and dendritic cells including cationic, mannose, Fc-targeted, CD11c-targeted and DC-SIGN targeted liposome carriers. These have had various degrees of success, but often have the drawback of requiring complex targeting molecules and antibodies that present challenges to large scale production and storage. An exception is the mannose targeting system which utilizes a mannose sugar to target the macrophage mannose receptor and is a robust and simple system. Cationic liposomes appear attractive for targeting cells, but when injected systemically into the body, they aggregate and accumulate almost entirely in the lung and liver. While many systems have been developed to target macrophages and dendritic cells, there are few available options for the targeting of MDSCs, neutrophils and B cells. To overcome these shortcomings and challenges, a system has been designed that utilizes the patient's own endogenous complement C3. The liposomes bind to C3 after injection, resulting in targeting to cell types that have the receptor for C3. The system utilizes small molecules, which would allow for scaling up and storage, binds to endogenous C3 which would cut down on toxicities, and targets a wide variety of immune cells that regulate the antitumor immune response.

Complement C3 is a protein that is present in normal human blood and is activated in the presence of a pathogen or foreign molecule. When activated, the disulfide bond between the alpha and beta strand of the complement protein is cleaved to give the active form, C3b. The activated fragments bind to pathogen surfaces which are then recognized by innate immune cells for phagocytosis, destruction and antigen presentation. The described liposome system contains lipid bound to an orthopyridyl disulfide (OPSS) moiety, which forms a disulfide bond with the exposed sulfhydryl group of activated C3 protein present in normal serum. OPSS-liposomes coated in C3 proteins are targeted for phagocytosis by the innate immune system. In a previous study, it was shown that liposomes containing OPSS bind to complement C3 in mouse serum, resulting in uptake by immune cells that have the receptor for complement. Upon systemic administration into tumor bearing mice, liposomes were taken up by MDSCs which infiltrated the spleen and tumor.

One goal of the current study was to establish if the liposomes also bound to human complement C3 and if so, to fully characterize the cell types in human blood that engulf C3 bound liposomes using flow cytometry analysis. Possible uses of complement C3 bound liposomes include delivery of tumor antigen or stimulating molecules to APCs, and delivery of drugs that can reprogram immunosuppressive MDSCs, macrophages and neutrophils to an immunostimulatory phenotype.

2. Materials and Methods

i. Reagents

1,2-dipalmitoyl-sn-glycero-3-phosphocholine (DPPC), 1,2-distearoyl-sn-glycero-3-phosphocholine (DSPC), 1,2-distearoyl-sn-glycero-3-phosphoethanolamine-N-[poly(ethylene glycol)-2000] (DSPE-PEG(2000)), and 1,2-distearoyl-sn-glycero-3-phosphoethanolamine-N-[PDP-poly(ethylene glycol)-2000] (DSPE-PEG(2000)-PDP) used for liposome preparation were purchased from Avanti Polar Lipids (Alabaster, Ala.). Fluorescently tagged lipid, Lissamine rhodamine B 1,2-dihexadecanoyl-sn-glycero-3-phosphoethanolamine (RhoPE), was purchased from Life Technologies (Grand Island, N.Y., USA). Size exclusion chromatography utilized CL-4B Sepharose gel, purchased from Sigma-Aldrich (St. Louis, Mo., USA). Red blood cell lysis buffer was purchased from eBioscience (San Diego, Calif., USA). Goat anti-human complement C3 was obtained from MP Biomedicals (Solon, Ohio, USA). Secondary donkey anti-goat 800 IgG was purchased from Li-Cor Bioscience (Lincoln, Nebr., USA). Normal human serum complement and C3-depleted human serum were obtained from Quidel Corporation (Athens, Ohio, USA). Flow cytometry antibodies, PE/Cy7 anti-human CD16, Brilliant Violet 605 anti-human CD33, Brilliant Violet 650 anti-human CD20, Brilliant Violet 785 anti-human CD56 (NCAM), were purchased from BioLegend (San Diego, Calif., USA). Flow cytometry antibodies, APC-Alexa Fluor 700 anti-human CD11c, APC-Alexa Fluor 750 anti-human CD11b, PC5.5 anti-human HLA-DR, FITC anti-human CD45, ECD anti-human CD3, Pacific Blue anti-human CD15, and APC anti-human CD14, were purchased from Beckman Coulter (Brea, Calif., USA). All other chemicals and reagents were purchased from Thermo Fisher Scientific (Pittsburgh, Pa., USA).

ii. Liposome Preparation

Liposomes were prepared using the film hydration-extrusion method as previously described 7,14. Liposomes containing DSPE-PEG(2000)-PDP are referred to as OPSS-liposomes; liposomes containing DSPE-PEG(2000) are referred to as control-liposomes. To produce OPSS-liposomes, DPPC/DSPC/DSPE-PEG(2000)-PDP/RhoPE in chloroform were briefly mixed at a molar ratio of 83:11:5:1). For control-liposomes, DSPE-PEG(2000) was substituted for DSPE-PEG(2000)-PDP to maintain the same ratio of DSPE-PEG. Lipids were dried under a nitrogen stream for 1 hour to remove any chloroform residue. The lipid film was rehydrated in 0.7 mL of filtered water, and extruded 9 times through a 200 nm polycarbonate membrane filter at 47° C. Liposomes were column purified using a CL-4B Sepharose column hydrated in 1× PBS, pH 7.4. Liposome fraction was diluted to a concentration of 0.875 mg lipid/mL. The amount of OPSS-liposome and control-liposome in each sample was normalized using a NanoDrop 2000 UV-Vis Spectrophotometer. Liposome size was obtained using a Malvern zetasizer Nano-S (Malvern Instruments, Malvern, UK). Control-liposome diameter was measured as 141.8±47.29 nm, and OPSS-liposome diameter was 140.4±43.76 nm.

iii. Liposome Binding of Activated C3

SDS-PAGE and Western blot techniques were used to determine if liposomes bind activated C3 when exposed to complete human serum. A 1:1 sample of OPSS-liposomes or control-liposomes with human serum was incubated for 1 hour at 37° C. Liposomes were isolated from the serum by centrifuging in Beckman 5×41 mm ultra-clear tubes in a SW50.1 rotor at 200,000×g for 10 minutes at 4° C. in a Beckman L8-70 ultracentrifuge. Liposomes were centrifuged and rinsed three times in 1× PBS before being rehydrated in 1× PBS. Samples were mixed 1:1 with a 2× reducing sample loading buffer and heated at 95° C. for 4 minutes. Samples were then run on a precast 10% SDS-PAGE gel (Bio-Rad Laboratories) for 1 hour at 120 volts. After electrophoresis was complete, the gel was soaked in transfer buffer (25 mM Tris-base, 192 mM glycine) for 15-20 minutes to equilibrate before transfer. The proteins were then electroblotted onto Immobilon PVDF membrane (Sigma-Aldrich) at 12 volts overnight. Total proteins associated with the liposomes were identified by colloidal gold staining of the blot. C3 proteins associated with the liposomes were detected with goat anti-human complement C3 and secondary donkey anti-goat 800 IgG, and visualized with a Li-Cor infrared scanner with Odyssey software.

iv. In Vitro Uptake of Liposomes

An in vitro analysis of liposome uptake was performed to determine which cell types take up liposomes in human blood. Peripheral blood mononuclear cells (PBMCs) were isolated from whole blood obtained from 5 healthy human volunteers. Immediately after drawing, the blood was incubated in red blood cell lysis buffer for 10-15 minutes. The samples were then centrifuged at 500×g for 5 minutes in an Eppendorf 5804 centrifuge. Samples were rinsed in 1× PBS and resuspended in RPMI media. Cells were aliquoted into a 96-well V-bottom plate with 80 μL per well to achieve a concentration of approximately 160,000 cells per well (2×106 per mL). OPSS-liposomes and control-liposomes were incubated for 1 hour at 37° C. with an equal volume of normal human serum or serum that had been depleted of complement C3. Twenty μL of the liposomes+serum sample was added to the 80 μL of cells in each well to bring the final volume in each well up to 100 μL with a concentration of 10% serum. Cells were exposed to liposomes for 2 hours before collection and analysis by flow cytometry.

v. Flow Cytometry Analysis

Cells were analyzed by flow cytometry to determine the populations of cells that were positive for rhodamine-labeled liposomes. Collected cells were centrifuged in a 96-well V-bottom polystyrene microplate at 2000 rpm in a Sorvall T6000D centrifuge for 3 minutes and resuspended in 100 μL FACS buffer (1× PBS+1% BSA) containing 1 μL each of anti-human antibodies against CD45, CD3, HLA-DR, CD16, CD14, CD11c, CD11b, CD15, CD33, CD20, and CD56. Cells were incubated in the dark with the staining buffer at 4° C. for 20 minutes. After staining, cells were centrifuged as above and resuspended in 200 μL of FACS buffer and analyzed using a Beckman Coulter CytoFLEX flow cytometer with CytExpert software. After gating to find cell populations, the percentage of rhodamine-liposome positive cells was determined, averaged for the 5 patients, and presented as mean±SE (n=5).

vi. Fluorescent Microscopy

Cells were treated for 2 hours with OPSS- or control-liposomes that had been incubated in complement C3-containing or depleted human serum as described above. Cells were centrifuged at 500×g for 5 minutes and rinsed twice with PBS before resuspension and transfer to a flat bottom Falcon microtest 96-well assay plate, black/clear bottom (Becton Dickinson Labware, Franklin Lakes, N.J., USA). Cells were imaged with a Leica DMI6000B inverted fluorescence microscope (Leica Microsystems, Buffalo Grove, Ill., USA), and photos were taken using a 10×objective utilizing Leica Application Suite, version 3.7.0 software.

3. Results

i. OPSS-Liposomes Bind Complement C3 in Normal Human Serum

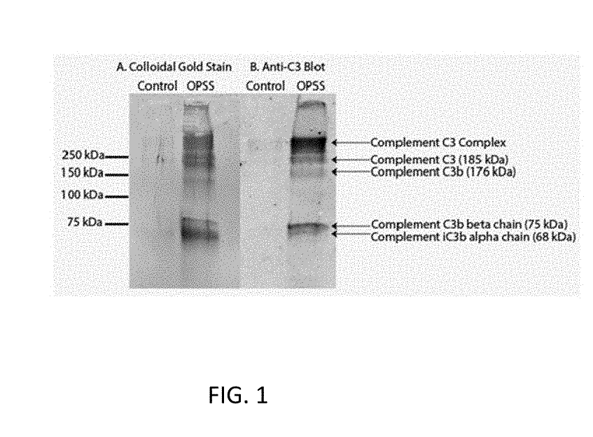

The ability of OPSS-liposomes to bind complement protein C3 was determined by SDS-PAGE and Western blot analysis (FIG. 1). When complement C3 protein in normal serum is activated, the disulfide bond between the alpha and beta chain of the protein is cleaved, leaving an exposed sulfhydryl group on the activated C3b fragment. The OPSS group on the PEGylated lipid binds to these activated fragments and forms the C3 bound liposomes (C3-liposomes), which are taken up by cells that have receptors for activated complement C3. OPSS-liposomes and liposomes lacking the OPSS group (control-liposomes) were incubated in normal human serum containing all the complement proteins to test the specificity towards complement C3, one of the most abundant complement proteins in serum. After incubation with serum, the liposomes were pelleted by centrifugation and rinsed to remove the serum before analysis by SDS-PAGE gel electrophoresis and Western blot. Control-liposomes lacking the OPSS group did not bind complement C3, while the OPSS-liposomes were shown to attach complement C3 and its activated fragments (FIG. 1). A duplicate Western blot was stained with colloidal gold, to detect all proteins that were associated with the liposomes. The overlap in bands between the colloidal gold stain and the anti-C3 blot shows that binding of C3 to the liposomes with the OPSS group is specific and does not occur in control liposomes without the OPSS group.

ii. White Blood Cells Internalize OPSS-Liposomes

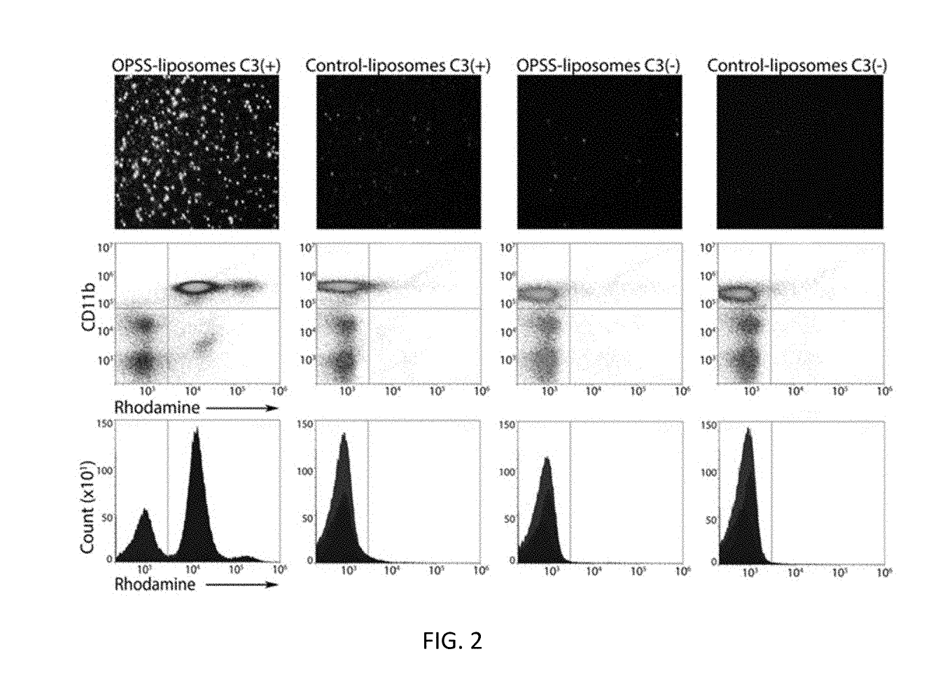

OPSS- and control-liposomes were incubated in human serum that either had functional complement C3 (C3+) or was depleted of complement C3 (C3−), and these liposomes were then administered to white blood cells isolated from human blood. Uptake of liposomes into cells was observed via fluorescence of rhodamine attached to a lipid incorporated into the liposomal membranes. Members of the complement receptor family that are found on white blood cells include complement receptors 1, 2, and 3 (CR1, CR2, and CR3). CR3 can be identified by the surface marker, CD11b, and is the major complement receptor of the myeloid cell populations. These receptors are expressed on the surface of cells and bind and internalize particles attached to complement proteins. Fluorescent microscopy and flow cytometry analysis showed that OPSS-liposomes incubated in C3-containing serum were readily taken up by CD11b+ cells (70.38%), while control-liposomes showed very little uptake (2.39%) (FIG. 2). OPSS-liposomes incubated in serum depleted of complement C3 also displayed little internalization into CD11b+ cells (OPSS: 1.08%, Control: 0.81%) demonstrating the importance of both the OPSS group and the bound activated C3 protein in the targeting mechanism (FIG. 2). The CD11b-negative cells that had taken up OPSS-liposomes in C3-positive serum (lower right quadrant of left panel, FIG. 2) were identified as B cells (data not shown), known to contain CR2 receptors that can bind activated complement C3 fragments.

iii. Liposome Uptake by Myeloid Derived Suppressor Cells

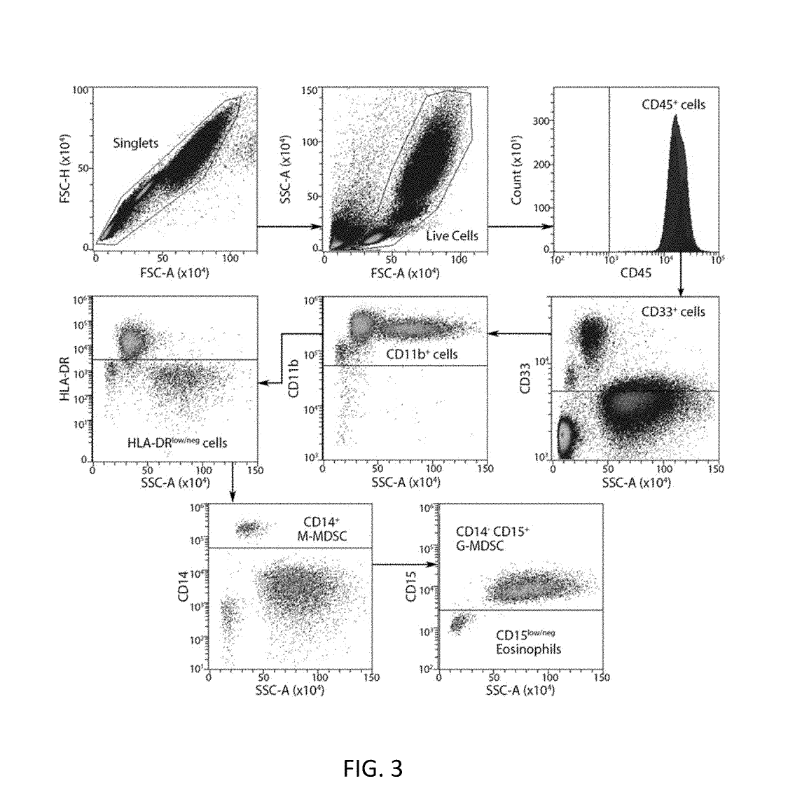

MDSCs are a heterogeneous population of cells that contain several identifying cell surface markers. These cells also express complement receptor CR3 (CD11b+), enabling C3 bound OPSS-liposome to target both monocytic MDSC (M-MDSC) and granulocytic MDSC (G-MDSC). Normal human white blood cells were stained with antibodies against several cell surface markers to identify the MDSCs by flow cytometry. Monocytic myeloid derived suppressor cells (M-MDSC) were detected according to their cell surface marker phenotype: CD33+/hi, CD11b+, HLA-DR−/low, and CD14+/hi. Granulocytic myeloid derived suppressor cells (G-MDSC) were determined by: CD33+/low, CD11b+, HLA-DR−/low, CD14−, and CD15+ (FIG. 3). The gating strategy used to distinguish M-MDSC and G-MDSC can be seen in FIG. 3.

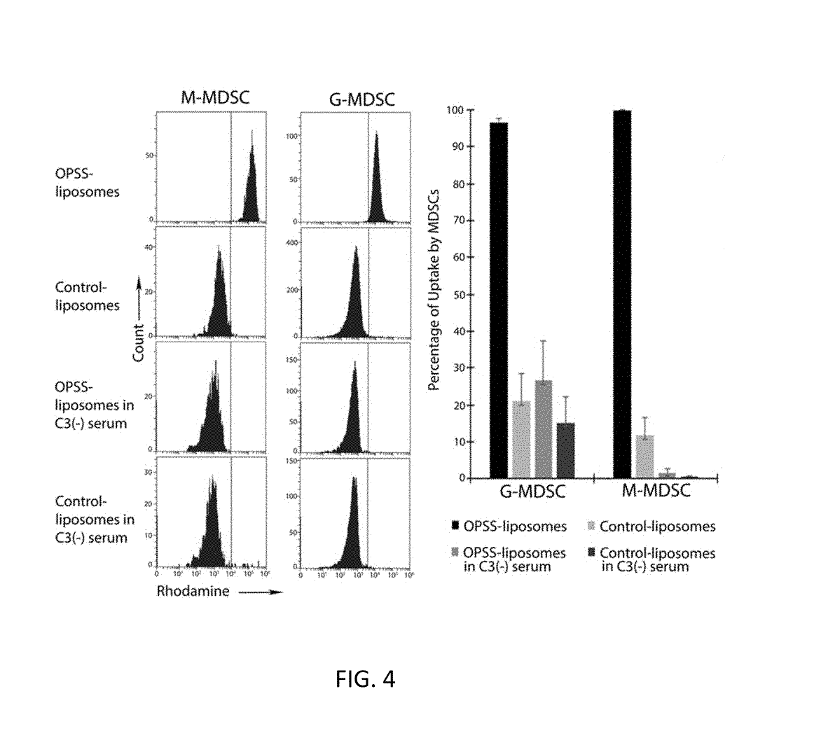

Human WBC were exposed to rhodamine-labeled liposomes that had been incubated in C3-positive versus C3 depleted serum to determine if bound C3 led to internalization of OPSS-liposomes. Both M-MDSC and G-MDSC showed high internalization of C3-bound OPSS-liposomes with 99.8±0.1% and 96.7±0.9% of cells taking up liposomes, respectively. Control-liposomes and OPSS-liposomes incubated in serum depleted of complement C3 showed significantly reduced uptake, with G-MDSC showing less than 27% uptake and M-MDSC showing less than 12% uptake in all conditions (FIG. 4). Neutrophils that were CD15+, but did not display immature G-MDSC markers also took up C3-bound OPSS liposomes (77±7%), with less than 3% of cells taking up liposomes if serum was depleted of C3 or OPSS wasn't present (data not shown).

iv. Liposome Uptake by Antigen Presenting Cells

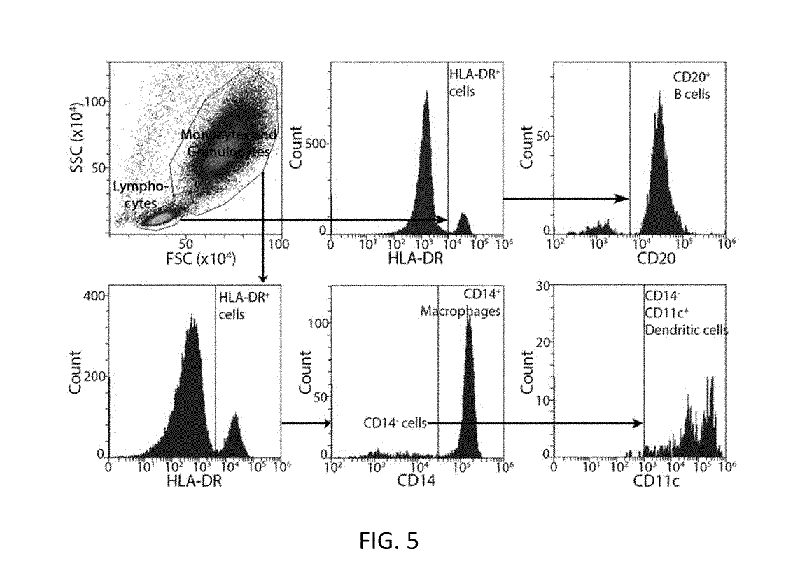

Antigen presenting cells (macrophages, dendritic cells, and B cells) were identified by flow cytometry and analyzed for uptake of rhodamine-labeled liposomes. Single cells were first selected that were positive for the common leukocyte antigen, CD45. These cells were then selected by size and internal complexity (SSC vs FSC) to separate the monocyte/granulocyte population from the lymphocytes (FIG. 5). B cells were identified from the lymphocyte population by the surface markers HLA-DR and CD20. Macrophages were identified from the monocyte/granulocyte population by the presence of HLA-DR and CD14 surface marker expression. Myeloid dendritic cells were isolated from the same population by expression of HLA-DR, low expression of CD14, and expression of CD11c (FIG. 5).

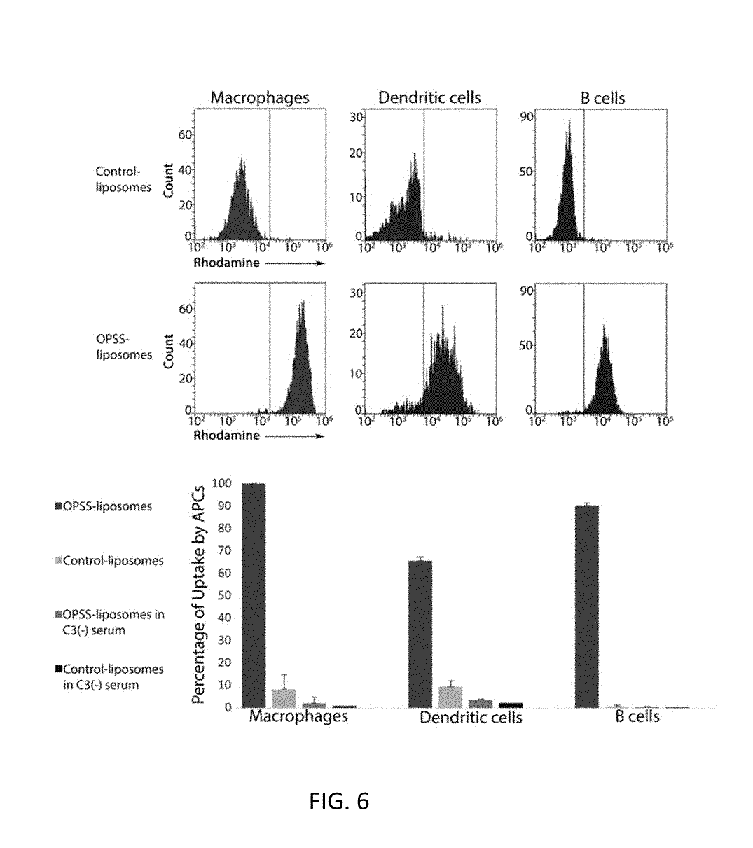

Antigen presenting cells displayed selective uptake of rhodamine-labeled OPSS-liposomes that had been incubated in C3-positive serum and were therefore bound to complement C3. OPSS-liposomes incubated in C3 depleted serum and control-liposomes incubated in C3-positive or C3 depleted serum showed little uptake by APCs (FIG. 6). OPSS-liposomes and control-liposomes incubated in C3-positive serum showed internalization into 99.99±0.01% and 8±1% of macrophages, respectively. This demonstrates the ability of the C3 bound OPSS-liposomes to enhance the natural liposomal clearance by phagocytic macrophages and specifically target the complement receptors for uptake. 66±7% of myeloid dendritic cells internalized OPSS-liposomes, while only 10±3% internalized control-liposomes after incubation in C3-positive serum. B cells displayed a similar pattern with 90±2% uptake of OPSS-liposomes compared to 0.8±0.2% uptake of control-liposomes. When liposomes were incubated in C3 depleted serum, uptake was seen in less than 4% of APCs for both OPSS- and control-liposomes (FIG. 6). These results show that both the OPSS group and the presence of complement C3 drive uptake of liposomes into the APCs: dendritic cells, macrophages and B cells.

v. Liposome Uptake by Lymphocytes

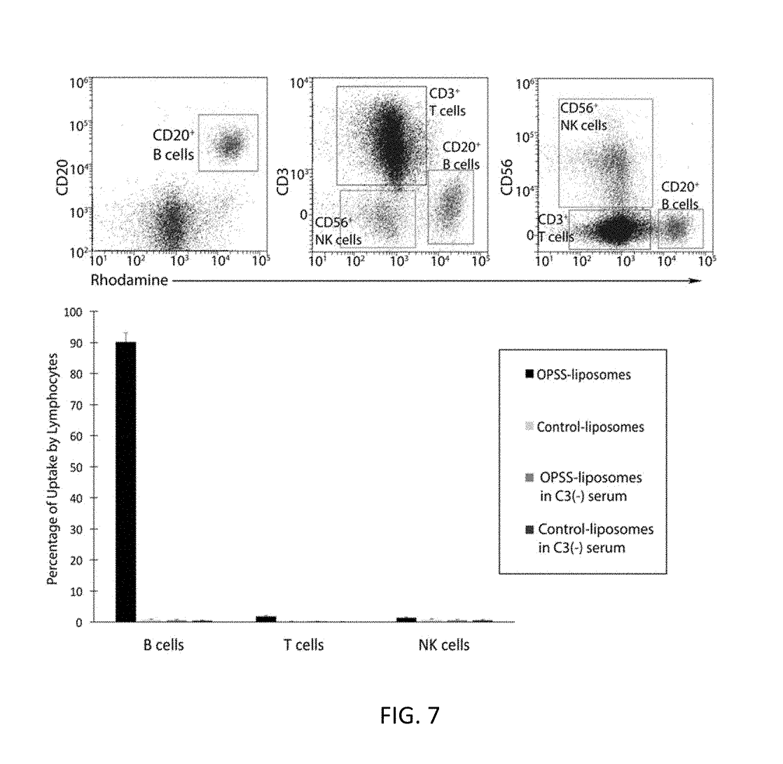

T cell, NK cell and B cell populations were analyzed for their uptake of rhodamine-labeled liposomes. The lymphocyte population was initially selected as positive for CD45, and by size and internal complexity (SSC vs FSC). This population was further broken down to identify CD20+ B cells, CD3+ T cells and CD56+ NK cells. The T cell and NK cell populations showed minimal uptake of OPSS-liposomes and control-liposomes incubated in either C3-positive or C3 depleted serum with less than 2% of T and NK cells positive in all conditions. In contrast, OPSS-liposomes incubated in C3-positive serum were taken up by 90±2% of B cells, while less than 3% of B cells took up OPSS-liposomes incubated in C3 depleted serum or control-liposomes incubated in C3-positive or C3 depleted serum (FIG. 7). These data show that among the different leukocyte populations, B cells are the only population targeted by OPSS-liposomes bound to C3.

4. Discussion

The immune response against cancer is regulated by immune cells, many of which display the receptor for complement. Strategies for promoting an antitumor immune response would benefit from a nanoparticle system that can target these cells 3,9,16. Liposomes were therefore formulated with a lipid-attached OPSS group, which is capable of forming a disulfide bond with activated complement C3. After binding C3, these liposomes are taken up by human macrophages, M-MDSCs, G-MDSCs, neutrophils, dendritic cells and B cells, all of which display receptors for various complement C3 fragments. By utilizing this targeting mechanism, the C3-bound OPSS-liposomes should allow the delivery of tumor antigen or immunostimulatory drugs to these cell types.

Complement C3 is a component of the blood that is activated to C3b, revealing a thioester group capable of forming a disulfide bond with OPSS. Western blot analysis reveals that incubation of OPSS-liposomes in serum for 1 hour allows conjugation of C3b to the liposomes and that this binding is relatively specific with little other protein attached. C3b targets the complement CR1 receptor but can be further metabolized to iC2b and C3dg, which can target CR2 (iC3b, C3dg), CR3 (iC3b), CRIg (iC3b) and CR4 (iC3b) receptors. Most of the cells targeted by C3 bound OPSS-liposomes have the CR3 receptor, including macrophages, neutrophils, dendritic cells and MDCS. However, B cells, which express the CR2 receptor also readily engulf liposomes, implying that the liposomes can also target through the iC3b or C3dg breakdown product. Indeed, the Western blot shows that, on the basis of molecular weight, iC3b is part of the complex that is conjugated to the liposomes. While all cells of myeloid lineage showed internalization of C3 bound OPSS-liposomes, presumably due to the presence of the myeloid CR3 (CD11b) receptor, the lymphocyte population of cells was limited in its uptake of liposomes, with the exception of B cells.

Myeloid derived suppressor cells (MDSCs) showed a high level of uptake of activated C3 bound OPSS-liposomes. When liposomes lacked the OPSS group (control-liposomes), there was little internalization by cells. Cells also did not take up OPSS-liposomes when serum was depleted of complement C3, demonstrating the importance of both the liposomal OPSS group and complement C3 for targeting. MDSCs are a heterogeneous population of immature cells that include granulocytic and monocytic subtypes. In cancer patients, the population of MDSCs expands in number in response to cytokines, such as GM-CSF, released from the tumor. The overall number of MDSCs correlates directly with cancer stage and level of metastasis. MDSCs are critical in creating the immunosuppressive conditions in the tumor microenvironment of cancer patients. Being able to target this cell population and reverse the suppression would significantly improve treatments and therapies. C3-bound OPSS-liposomes are able to target efficiently both the monocytic and granulocytic populations of myeloid derived suppressor cells in human blood, which allows for direct delivery to these important cell types. Reprogramming of MDSCs has been shown using all-trans retinoic acid (ATRA), vitamin D and CpG oligonucleotides, but techniques for specific delivery of these compounds are still lacking. The disclosed targeted liposomal system provides a means to target MDSCs and test different treatments, which could relieve the suppression and possibly revert these cells towards their non-suppressive phenotype.

The C3-liposomes are also taken up in a complement-dependent pathway by all three types of APCs: dendritic cells, macrophages and B cells. The first step in creating a robust adaptive immune response against cancer cells is efficient presentation of tumor antigen by APCs to the effector cells of the immune system. APCs present antigen to T helper cells via MHCII molecules. Additionally, dendritic cells and B cells, have been shown to cross present antigen via MHCI molecules, allowing for the stimulation of T killer cells. Techniques to improve on antigen presentation include ex vivo strategies such as adoptive T cell transfer and strategies such as nanoparticle antigen delivery. Ex vivo techniques are costly and have the drawback that they do not often create a memory T cell population after inoculation into the patient. Nanoparticle delivery systems have had some success, but often they are targeted only to macrophages and dendritic cells, and most targeted systems require costly antibodies or peptides that are difficult to store and scale up to pharmaceutical quantities. The advantage to using OPSS-liposomes is that OPSS is a small low-cost molecule that binds the patient's endogenous complement C3 and targets all three APCs, including B cells. OPSS-liposomes could encapsulate tumor antigen or activating oligonucleotides to improve antigen presentation to effector cells. In addition, to provoke a B cell antibody response, it is critical that B cells are stimulated through their complement receptor which lowers the stimulation threshold at which they produce antibody by approximately 1000-fold. By targeting antigen to all three APC cell types via the complement system, C3-liposomes could activate T cells and increase antibody production by B cells, leading to a robust and enduring antitumor immune response.

These experiments show a technique for targeting immune cells that play a key role in cancer progression, including MDSCs, neutrophils, macrophages, dendritic cells and B cells. These data were obtained using the blood of healthy human volunteers, and it is important to remember that the number and phenotypes of immune cells will presumably be different in cancer patients.

### B. Delivering Liposomes to All Three APCs Through Complement Driven Internalization

Tumor associated antigens (TAA) such as Melanoma Associated Antigen (MAGE) and Epithelial Tumor Antigen (ETA) are at the heart of immunotherapy as they provide the unique pattern that allows T cells and B cells to selectively target cancer cells. With the discovery of dozens of tumor antigens, the goal of many cancer immunotherapies including vaccines, adoptive T cell therapy, and chimeric antigen receptor T cells (CARs) has been to improve recognition of these antigens by the immune system. Creating a balanced immune response that involves T helper cell, cytotoxic T cell (CTL) and B cell immunity, while avoiding immune tolerance, requires antigen presentation that closely mimics an actual infection 6. In a significant step forward, a liposome system has been created that encapsulates tumor antigen and targets all three antigen presenting cell (APC) types, B cells, macrophages and dendritic cells. The liposomes are taken up through complement mediated pathways which activate the APCs, allowing for efficient antigen presentation and potent T cell stimulation.

Complement C3 is a component of the blood which binds to foreign pathogens in the body, marking them for uptake and destruction by the immune system. APCs display complement receptors on their surface, which allow them to recognize and engulf complement coated pathogens. Dendritic cells and B cells express Complement Receptor 2 (CR2) while macrophages and dendritic cells express Complement Receptor 3 (CR3). After being endocytosed, pathogens are broken down and pieces of them are displayed as antigen on major histocompatibility complex (MHC) II complexes which are recognized by T helper cells. Additionally, dendritic cells are capable of cross presenting antigen through MHCI complexes, leading to CTL stimulation. A combination of all three APCs displaying both MHCI and MHCII presentation is necessary to achieve a balanced and robust CTL, memory T cell and antibody mediated immune response. While nanoparticles have long been explored for their ability to deliver antigen to immune cells, the liposome system described herein is the first nanoparticle system that can bind selectively to complement and target all three APC cell types through complement mediated internalization.

Binding of complement receptors on APCs leads to increased activation which facilitates a strong immune response. If APCs are not properly activated, co-stimulatory molecules are not displayed on their surface, resulting in upregulation of T regulatory cells, immune evasion and tolerance to tumor antigens. Impressively, B cells stimulated through their CR2 receptor have a threshold for activation that is reduced up to 1000-fold, and CR2 knockout experiments show that complement mediated stimulation is necessary for an antibody response. Since the liposomes bind activated complement C3 and are taken up by the complement receptors, they should be highly stimulatory to B cells. In addition, it has been shown that monocyte-derived dendritic cells take up C3 liposomes resulting in stimulation and display of activation markers on their surface. The combined ability of these liposomes to target and activate the APCs along with their ability to encapsulate high levels of tumor antigen should allow for them to be a potent tool in creating anti-tumor immunity.

The most promising therapeutic approach for cancer immunotherapy involves targeting tumor induced immune suppression using multiple immunotherapeutic approaches. Tumors can evade the immune system by influencing the spectrum of infiltrating immune cells within the tumor and systemically. Combining immunotherapy strategies will allow for reversal of local and systemic immune suppression, tumor antigen presentation by APCs and elimination of primary and metastatic tumor cells through CTL, antibody and memory T cell antitumor response. Program Death-1 (PD-1) and cytotoxic T-lymphocyte-associated protein 4 (CTLA-4) inhibitors allow activated T cells to function within the tumor environment, but the immunotherapy field still lacks a dependable mechanism for activating a potent adaptive immune response within the patient. Using C3 targeted liposomes, antigens will be directly delivered to APCs both inside and outside the immune suppressive tumor microenvironment, creating robust CTL, memory T cell, and B cell immunity. This treatment would fill a gap in current immunotherapy and when used in conjunction with other strategies has the potential to enhance effector immune cell response to the tumor.

Strategies for improved antigen delivery include both in vivo and ex vivo techniques. In vivo techniques often utilize nanoparticles targeted to dendritic cells or macrophages in the body while ex vivo techniques remove dendritic cells and T cells, educate them with tumor antigens and then inject the cells back into the patient. Although ex vivo techniques can be effective, the disadvantages include the cost of individual treatment and the lack of memory T cell response because of the artificial conditions in which antigen is presented. A more practical and effective approach would be to deliver antigen in vivo where a robust and natural response can occur.

Current nanoparticle antigen delivery techniques include cationic, mannose, Fc-targeted, CD11c-targeted and CD-sign targeted liposome carriers. In addition, liposomes have been modified to be pH sensitive, fusogenic or activated by ultrasound to promote delivery of antigen to the cytoplasm of dendritic cells. All of these have had some level of success, but there are drawbacks to each of the techniques. Cationic nanoparticles are attractive because they bind to cell membranes which are negatively charged, but when injected systemically into the body, they aggregate and accumulate almost entirely in the lung and liver. Most of the actively targeted nanoparticles listed above use antibodies or fragments of antibodies to target dendritic cells. This not only limits delivery to dendritic cells, but the challenges of scaling up and storing such complicated liposomal formulations is daunting. Fusogenic and pH sensitive liposomes aim at cytoplasmic delivery with the goal of driving a CTL response. This could be effective, but a balanced response will also require MHCII cross presentation by dendritic cells and delivery to all three APCs. Of all the systems listed above, the mannose liposomes are the most attractive, because they use a small simple sugar to direct uptake by macrophages and dendritic cells. The advantage of this system is that the liposomes are targeted via complement C3 to the complement receptor which has been shown to be a potent APC activator. In addition, these C3-liposomes are the first system that can deliver to not only macrophages and dendritic cells, but to B cells as well. B cell antigen presentation is critical for CD4 T cell activation and may even be involved in cross presentation to CTLs. Targeting antigen to all three APCs should allow for a more effective and balanced immune response.

The liposomal system described herein combines practical simplicity with efficient delivery to all three types of APCs. The liposomes contain a lipid conjugated to a small disulfide forming group that is simple and cost effective. When injected into a mouse, the liposomes form a disulfide bond with activated C3 which displays an exposed sulfhydryl group. This binding is efficient and specific. With complement C3 displayed on their surface, liposomes are engulfed by all cells that display receptors for C3. Using human blood in vitro selective and high level of uptake into all three APCs, neutrophils and myeloid derived suppressor cells (MDSCs) has been shown. Unlike targeting systems that require ligand or antibody conjugation, this drug delivery system does not require complex chemistry and could be efficiently increased to pharmaceutically relevant quantities. The liposomes target the receptors for activated C3 present in both mice and humans, allowing for a smooth transition from animal experiments to human experiments. Finally, since the system uses the patient's own C3 protein, the liposomes should not display the immunogenicity and toxicity associated with injection of foreign antibodies and targeting ligands. Balanced and efficient antigen presentation is vital for a proper adaptive immune response. By delivering to all three APCs through complement driven internalization, this system has the potential to improve on currently available techniques for tumor antigen presentation.

1. Uptake of C3-Liposome In Vivo

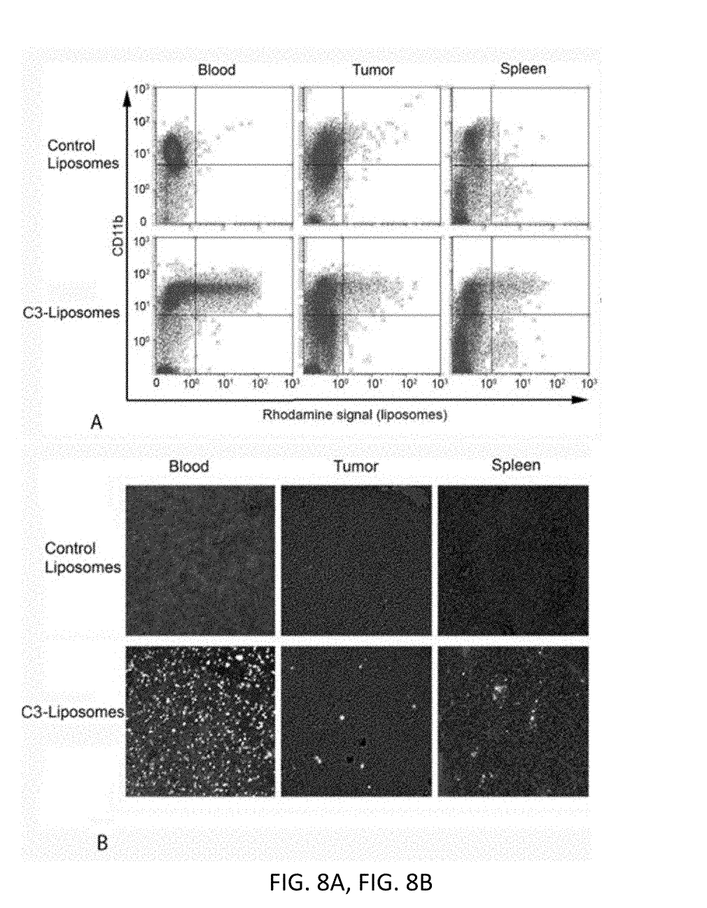

C3-liposomes that were delivered systemically to tumor bearing mice were specifically taken up by cells that display complement receptors. CD11b (receptor for activated C3) positive cells that had taken up liposomes were found in the blood, tumor and spleen (FIG. 8A). The conclusions from flow cytometry analysis were confirmed by histology of tissues from mice injected with C3- or control-liposomes. Mice injected with control-liposomes showed very little evidence of rhodamine labeled liposomes within the tissues, while mice injected with C3-liposomes had liposome-containing cells distributed in the blood, tumor, and spleen (FIG. 8B)

2. Antigen Delivery to APCs and T Cell Stimulation

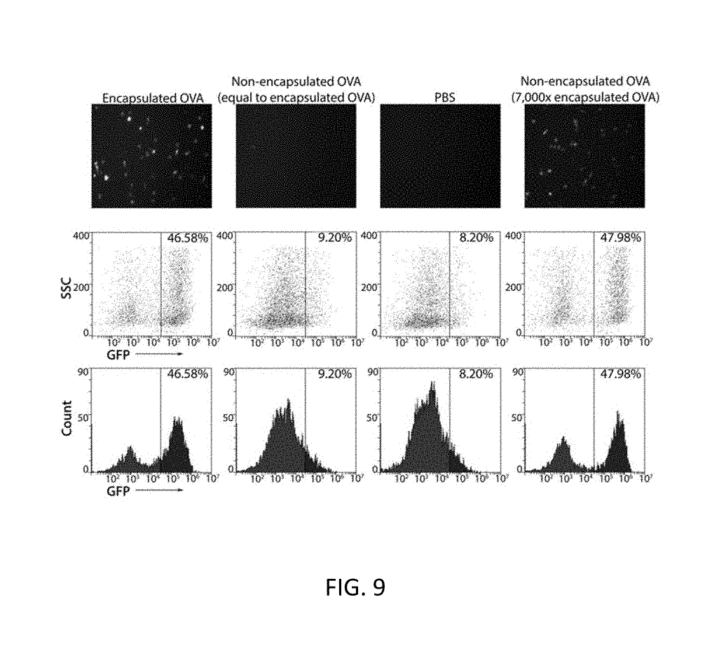

To derive dendritic cells from immature monocytes, bone marrow cells from mice were exposed for 6 days to the cytokines, GM-CSF and IL-4. The bone marrow derived dendritic cells were then exposed for 24 hours to C3-liposomes that had been loaded with ovalbumin (Ova C3-liposomes). Ovalbumin is commonly used as a model tumor antigen. To determine the effectiveness of antigen presentation, T cells that recognize ovalbumin through their T cell receptor and express GFP (Ova-GFP T cells) after being activated by APCs showing ovalbumin were used. This is a system that allows for rapid and accurate assessment of antigen presentation. After incubating dendritic cells with Ova C3-liposomes, Ova GFP T cells were added and incubated for 24 hours. Fluorescent microscopy shows that many of the T cells have expressed GFP, demonstrating that ovalbumin was presented to the T cells by APCs (FIG. 9). In comparison, when non-encapsulated ovalbumin was added in the same concentration to the bone marrow cells, there was little evidence of green T cells. In fact, 7000-fold as much non encapsulated ovalbumin was used to get the same T cell stimulation as when ovalbumin was loaded into C3-liposomes. Analysis by flow cytometry shows that 46.58% of T cells were activated with Ova C3-liposomes while only 8 to 9% were activated by PBS or control levels of non-encapsulated ovalbumin, demonstrating the efficiency of C3-liposomes for antigen delivery.

3. Activation of Dendritic Cells

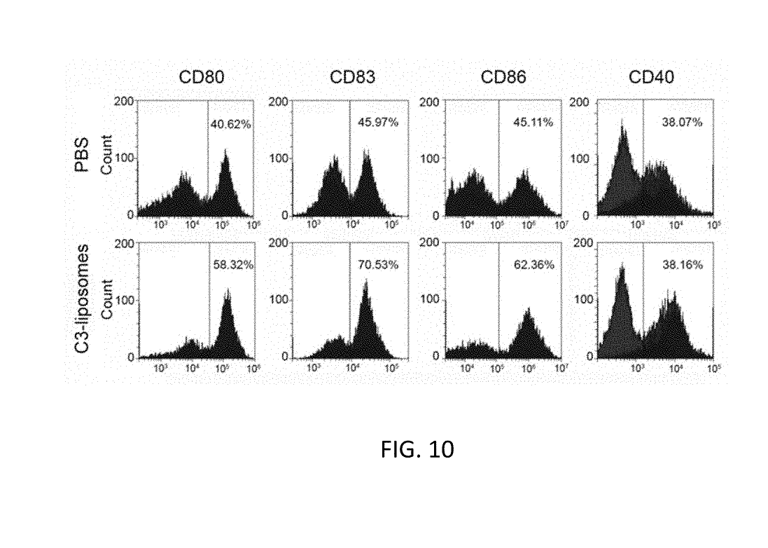

The literature shows that B cells are highly activated when complement binds to the CR2 receptor, but there are less data available on the activation of dendritic cells in response to complement C3 binding. To determine if C3-liposomes activated dendritic cells, bone marrow derived dendritic cells were prepared as described above and exposed them to C3-liposomes for 48 hours. After this period, dendritic cells were analyzed by flow cytometry for activation markers including CD40, CD80, CD83 and CD86. Compared to PBS and control-liposomes, there is a clear increase in the activation markers in response to uptake of C3-liposomes (FIG. 10). This activation of dendritic cells is vital since antigen presentation by dendritic cells not displaying costimulatory molecules can lead to T cell tolerance and immunosuppression. The ability of C3-liposomes to activate dendritic and B cells combined with their ability to deliver high levels of antigen to APCs, should allow for a robust anti-tumor immune response.

4. Experimental Design

i. A20-ova Tumor Mouse Model

The tumor model proposed for these experiments is based on the A20-ova cell line, a mature B-cell lymphoma that expresses ovalbumin and forms tumors in Balb/c mice. This model is well established for determining the effectiveness of tumor antigen delivery. Since transfected ovalbumin DNA is expressed by A20-ova cells, mice that have been successfully vaccinated with ovalbumin show reduced tumor growth. A20 cells are immunogenic, responding to immunotherapy and showing decreased tumor growth after treatment with immune checkpoint inhibition, allowing for experiments that test combination immunotherapies. Another advantage of this tumor model is that ovalbumin is readily available at low cost, permitting freedom to optimize liposomal formulations and repeat experiments without financial limitation. Finally, earlier work has optimized ovalbumin containing C3-liposomes and A20-ova tumors can provide continuity from in vitro work through the in vivo experiments. Although the A20 lymphoma line is B cells, earlier results do not indicate that C3-liposomes are taken up to a greater degree than control-liposomes. There are several other tumor antigen models including a B16 melanoma cell line that expresses MART-1 tumor antigen and a transgenic mouse that expresses human epidermal growth factor receptor 2 (Her-2).

5. Characterize Tissue and Cellular Biodistribution of C3-Liposomes After Injection into Tumor Bearing Mice.

i. Objective:

Studies show that C3-liposomes are taken up by human dendritic cells, macrophages and B cells in whole blood in vitro and that when injected systemically into tumor bearing mice, they are taken up by cells that have complement receptor 3 (Cd11b+ cells). These experiments can fully characterize the cell and tissue biodistribution of C3-liposomes in tumor bearing mice. Characterization of cell populations by flow cytometry and tissue sections by immunohistochemistry (IHC) can be performed on mouse whole blood and tissue to quantitate C3-liposome uptake in immune cells of the blood, tumor, spleen and lymph nodes.

ii. Design:

Balb/c mice with A20-ova tumors can be established by injecting 1×10̂6 cells subcutaneously into the right and left flank. After 2 weeks, when tumors are approximately 200 cm³, C3-liposomes that are labeled with a fluorescent rhodamine tag can be injected. In addition, control liposomes that do not have the OPSS group and therefore do not bind activated C3, can be utilized as a control.

iii. Experimental and Control Conditions:

There can be 4 groups of mice with 6 mice in each group receiving the following treatment. 1: C3 liposomes via tail vein injection. 2: Control-liposomes via tail vein injection. 3: C3-liposomes via peritumoral injection. 4: Control-liposomes via peritumoral injection. The sample size, six, in the study was determined by power analysis at 5% significance level and 80% power was performed. Assuming 50% variation in the data, to observe a 100% difference would require a sample size of six for each group.

iv. Analysis of Liposome Uptake:

Cells and tissue can be collected 3 hours after injection with fluorescently tagged liposomes. The spleen, liver, lymph nodes and tumor can be divided and placed in Hank's buffered saline solution (HBSS) for flow cytometry, preserved in 10% Neutral Buffered formalin (NBF) for IHC, or flash frozen. Tissue placed in HBSS, can be digested using collagenase and prepared for analysis by flow cytometry (detailed methodology below). Flow cytometry with 13-color BD Cytoflex can be performed using surface markers (listed in methodology), which can allow quantification of the uptake of liposomes into B cells, T cells, NK cells, dendritic cells, macrophages, MDSCs and neutrophils in the blood, spleen, liver, tumor and lymph nodes. The tissues preserved in NBF, can be processed and sectioned for IHC staining using antibodies against CD20 (B cells), F480 (macrophages), CD11c (dendritic cells) and CD3 (total T cells), to determine the uptake of liposomes and to observe their distribution and interaction with APCs and other stromal cells within the tissue. Detailed methodology for liposome preparation, tissue digestion, flow cytometry and IHC are described below.

v. Expectations:

Based on results on human whole blood that show B cell, macrophage, dendritic cell, neutrophil and MDSC uptake of C3-liposomes, these same cell types can engulf C3-liposomes in vivo, in the A20-ova mouse model. Although 90-95% of these cell populations have taken up C3-liposomes in vitro, in vivo, liposome positive cell populations can be closer to 15-25%. Initial IHC stains show that CD11b+ cells have taken up C3-liposome in the spleen, blood and tumor. C3-liposomes can also be taken up by APCs in the lymph node and interaction of liposome positive B cells, macrophages and dendritic cells can be seen with the T cells in both the spleen and lymph nodes.

vi. Methodology:

a. Liposome Preparation:

To create OPSS-liposomes, the lipids 1,2-Dipalmitoyl-sn-glycero-3-phosphocholine (DPPC), 1,2-distearoyl-sn-glycero-3-phosphocholine (DSPC), 1,2-dipalmitoyl-sn-glycero-3-[phospho-rac-(1-glycerol)] (DPPG), Lissamine rhodamine B 1,2-dihexadecanoyl-sn-glycero-3-phosphoethanolamine (RhoPE), 1,2-distearoyl-sn-glycero-3-phosphoethanolamine-N-[poly(ethylene glycol)-2000] (DSPE-PEG(2000)) and 1,2 distearoyl-sn-glycero-3-phosphoethanolamine-N-[PDP-poly (ethylene glycol)-2000] (DSPE-PEG(2000)-PDP) are suspended in chloroform and mixed at a molar ratio of 80:10:3:1:1:5 respectively. The solution is dried under a stream of nitrogen. The dried lipids are resuspended in 0.7 ml H2O that contains 160 mg/ml ovalbumin at 47° C. and then extruded through a 200 nm polycarbonate filter 7 times. The resulting liposomes have a diameter of 167±92 nm as determined by differential light scattering.

b. Tissue Digestion and Whole Blood for Flow Cytometry Analysis:

The spleens and tumors from tumor bearing mice injected with liposomes can be digested with collagenase at 37° C. for 20 minutes. Digestion mixtures can be quenched using RPMI containing 10% FBS and filtered through a 70 μm nylon strainer. The tumor, spleen and whole blood samples can be centrifuged and resuspended in red blood cell lysis buffer for 10 mins. After the red blood cells have been lysed, the remaining cells can be centrifuged and resuspended in FACS buffer for flow cytometry analysis.

c. Flow Cytometry:

Cells can be stained in a 96 well plate for 20 min at 4° C. with the following antibodies purchased from BioLegend: CD3, CD11b, CD11c, CD14, CD15, CD16, CD20, CD33, CD45, CD56 and HLADR. Liposomes can be labeled with a rhodamine conjugated lipid as previously above. The cells can be centrifuged, rinsed in FACS buffer and analyzed using a 13-color Beckman Dickinson cytoflex, equipped with Kaluza analysis software. Cell types can be determined according to their surface markers as follows: M-MDSC (CD11b, CD33, CD14, CD45, HLADRlo), G-MDSC (CD11b, CD33, CD14lo CD15, HLADRlo), Macrophage (CD14, CD45 CD11b, HLADR, CD11clo), Neutrophil (CD11b, CD33, CD1410 CD15, CD49lo, HLADRlo), Dendritic Cell (CD11c, CD11b, CD14, HLADR, CD45), T cell (CD3, CD45), B cell (CD20, CD19, CD45, HLADR, CD3 lo), NK cell (CD11b, CD56, CD45).

d. Immunohistochemistry:

Tissue can be fixed in 10% NBF for 24 hours then placed in 70% ethanol before being processed and paraffin embedded into tissue blocks. Serial 5 um sections can be cut using a Leica microtome. Tissue sections from the liver, spleen and tumor can be stained using the following antibodies: CD20 (B cells), F480 (macrophages), CD11c (dendritic cells) and CD3 (total T cells). The number of positively stained cells peritumorally and intratumorally can be counted in eight continuous non-overlapping fields at ×400 magnification and expressed as positive cells per high power field (c/hpf). IHC staining can be used to determine the uptake of liposomes in tissue and to observe liposome distribution and interaction with APCs and other stromal cells within the tissue.

6. Determine if Antigen Loaded C3-Liposomes Stimulate a T Cell- and B Cell-Response Using the A20-Ova Mouse Model.

i. Objective:

C3-liposomes can be effective in achieving a balanced adaptive immune response in tumor bearing mice. C3-liposomes loaded with ovalbumin can be delivered to A20-ova tumor bearing mice, and after 10 days, blood and tissue can be analyzed for presence of anti-ovalbumin antibodies and T cells.

ii. Design:

Balb/c mice with A20-ova lymphoma tumors can be established as described above. After 1 week, mice can be injected every other day via tail vein or peritumoral injection with Ova C3-liposomes, control liposomes or PBS. After 10 days, mice can be sacrificed and whole blood, spleen and tumor can be collected. Cells isolated from whole blood can be analyzed for the presence of IgG antibody using ELISA techniques to determine if a humoral B cell response to the tumor antigen ovalbumin was stimulated by Ova C3-liposomes. T cells can be collected from the tumor and spleen and cultured with ovalbumin bound stimulation beads for 3 days. Presence of ovalbumin reactive T cells can be determined by measuring T cell proliferation, IFN-γ levels and T cell surface activation markers. In addition, tumor infiltrating T cells can be quantified using flow cytometry and IHC to determine the presence of CD4 and CD8 positive T cells in tumor tissue.

iii. Experimental and Control Conditions:

There can be 3 groups of mice with 6 mice in each group receiving the following treatment via systemic tail vein injection. 1: Ova C3-liposomes. 2: Non-encapsulated ovalbumin at the same concentration as in group 1. 3: PBS. Additionally, the experiments can be run in parallel with the same experimental groups but with peritumoral injection near the tumor region to determine if localized immunization is more effective in creating an anti-ovalbumin immune response.

iv. Expectations:

In vitro experiments show that Ova C3-liposomes are engulfed by dendritic cells and are efficient stimulators of ovalbumin reactive T cells. In vivo administration of Ova C3-liposomes can initiate a potent anti-ovalbumin adaptive immune response compared to ovalbumin administered alone. Since C3-liposomes are taken up and activate APCs, the adaptive immune response can include both antibodies and T cells that recognize ovalbumin.

v. Methodology:

a. T Cell Antigenic Response:

T cells collected from whole blood, tumor and spleen can be isolated by negative selection from tissue homogenates (as described above). Ova-IC beads can be prepared as previously described. Briefly, the IgG fraction from OVA-immunized rabbits (Sigma Aldrich) is collected using Hi-trap protein G-sepharose. The IgG bound beads are mixed with ovalbumin to create Ova-IC beads that are able to stimulate Ova-reactive T cells. Splenic T cells (3×10⁵ cells) can be cultured with Ova-IC beads in RPMI 1640 (Invitrogen) containing 10% FBS and 1% penicillin-streptomycin (Hyclone) in a 96-well plate. T cell proliferation can be measured using the cell trace violet cell proliferation kit (Life Technologies), while T cell activation can be determined measuring IFN-γ levels by ELISA. T cell activation markers can be analyzed by flow cytometry for surface markers, CD3, CD4, CD8, CD25 and CD69 to determine degree of activation.

b. B cell Antibody Response and T Cell IFN-γ by ELISA:

Ova-specific antibody titer can be determined using standard ELISA techniques. Collected serum from the experimental and control mice can be incubated in a 96-well ELISA plate using ovalbumin as a capture antigen (Jackson ImmunoResearch Laboratories). Level of ovalbumin antibody captured by the plate can be determined using a goat anti-mouse IgG horseradish peroxidase for readout. T-cell IFN-γ levels in supernatants collected from T cell assays, described above, can also be quantified by ELISA.

7. Evaluate if Antigen Delivery with C3-Liposomes Results in Reduced Tumor Growth When Used as a Monotherapy and in Combination with PD-1 Checkpoint Blockade.

i. Objective:

OVA C3-liposomes can provoke an immune response that reduces A20-ova tumor growth. This ovalbumin expressing cell line forms tumors in Balb/c mice, but shows reduced tumor growth if mice are successfully vaccinated against ovalbumin. C3-liposomes can be tested first as a monotherapy and then in combination with PD-1 checkpoint inhibitor to determine if treatment increases intratumoral T cell presence and further reduces tumor growth.

ii. Design:

Balb/c mice with A20-ova lymphoma tumors can be established as described above. Mice can be treated either by systemic tail vein injection or peritumoral injection depending on the results above, which can indicate the method of administration that was more effective in provoking an adaptive immune response. Beginning at day 7 after tumor cell inoculation or when tumors are palpable, mice can be injected every other day with Ova C3-liposomes or with control treatments (see below) over 3 weeks. Tumor size can be measured daily, and after 3 weeks, mice can be sacrificed and lung, liver, spleen and tumor tissue will be collected. Tumors can be analyzed by flow cytometry for the presence of CD4 and CD8 positive T cells. Lung, spleen and liver tissue can be analyzed for metastatic lesions upon removal.

iii. Experimental and Control Conditions:

There can be 4 groups of mice with 6 mice in each group receiving the following treatment. 1: Ova C3-liposomes. 2: Non-encapsulated ovalbumin at the same concentration as in group 1. 3: C3-liposomes that do not contain ovalbumin. 4: PBS. Additionally, the experiment can be run in parallel with the same experimental group treatments in combination with systemically administered antibody against PD-1 (anti-PD-1).

iv. Expectations:

Ova C3-liposomes can reduce tumor growth as a monotherapy and can be even more effective when combined with immune checkpoint PD-1 antibodies. A20 lymphoma cells are highly immunogenic and respond to anti-PD-1 immunotherapies, which makes them attractive for studying the potency of immunotherapeutic techniques. C3-liposomes improve on current antigen delivery techniques and should provoke and immune response against the tumor antigen, ovalbumin, transfected into the Ova-A20 cell line. Tumor immune avoidance is multifaceted, and immunotherapy can be most effective when used in combination with two or more strategies to overcome the different mechanisms of tumor induced-immune suppression and evasion. Using the Ova C3-liposomes to stimulate T cells in combination with a checkpoint inhibitor to remove inhibition of tumor infiltrating T cells could result in a synergistic reduction in tumor growth.

C. Intratumoral Delivery of Antigen with Complement C3-Bound Liposomes Eliminates Established Tumors in Mice

Tumor antigens are proteins that provide specific targets for CD8+ T cells (cytotoxic T lymphocytes: CTLs), allowing the immune system to distinguish cancer cells from noncancerous cells. Tumor antigens can be mutated peptides, expressed genes which are normally silent, cancer-germline antigens, which are only present on tumor cells, or viral epitopes, present on virus-associated tumors. Alternatively, they can be normal proteins expressed at a higher degree on tumor cells, but still present in normal tissue (overexpressed or differentiation antigens). Regardless of the type of antigen, antigenic activation is essential for the success of cancer immunotherapies.

The goal of a tumor vaccine is to improve T cell recognition of tumor antigens. Tumor vaccines can be derived from a single tumor antigen, antigenic epitope, or multiple antigens for a given tumor. Using multiple antigenic and immunogenic epitopes is advantageous due to the occurrence of immunoediting, whereby cancer cells limit the expression of certain antigens to hinder immune surveillance and allow for immune escape. The presence of multiple lineages of CTLs with receptors specific for different antigens creates a persistent attack on tumor cells, even in the presence of tumor-mediated antigen downregulation.

T cells must encounter a certain threshold of antigen presentation to overcome the natural tolerance mechanisms in place to prevent autoimmunity and acute inflammation. This is especially relevant when working with tumor antigens derived from over-expressed or differentiated antigen variants, due to their expression on normal tissue. Targeted liposome nanoparticles are an effective means of increasing the amount of antigen delivered as well as increasing the specificity of delivery to APCs. In addition, liposomes can encapsulate multiple antigens simultaneously to strengthen the immune response against a tumor. Other strategies for tumor vaccines often involve ex vivo proliferation and treatment of autologous dendritic cells (DCs), followed by re-infusion into the patient, akin to adoptive T cell transfers. Targeted liposomes represent a more auspicious avenue for vaccination since antigenic peptides can be delivered in vivo to APCs without the need for costly ex vivo culturing and re-infusion into the patient.

Many liposome systems have been developed to target antigen presenting cells, such as cationic, mannose, Fc-targeted, CD11c-targeted, and DC-SIGN-targeted. Most of these systems require complex targeting molecules, antibodies or cationic lipids and many are associated with high levels of toxicity. A liposome nanoparticle has been developed that utilizes neutral lipids and endogenous serum proteins, thereby reducing toxicity from cationic lipids and foreign proteins while decreasing expense associated with targeting antibodies and ligands. The liposomes are formulated to bind activated complement C3 proteins (C3-liposomes), which enable specific targeting to a range of immune cells that carry the receptor for complement C3. These receptors are expressed primarily by myeloid cells, including macrophages, dendritic cells, and neutrophils and by B cells. It was previously shown that C3-liposomes are internalized by all myeloid cell types, making it a unique delivery device to APCs.

In this study C3-liposomes were assessed for their potential as a tumor vaccine. Using ovalbumin (OVA) as a model antigen, the ability of C3-liposomes to deliver antigen and activate T cells was tested in vitro with the reporter D011.10 T cell line. These studies were followed by in vivo experiments in the A20-OVA mouse model, where C3-liposomes were shown to deliver tumor antigen, activate an antigen-specific immune response and eliminate established tumors in mice.

1. Materials and Methods

i. Reagents

1,2-dipalmitoyl-sn-glycero-3-phosphocholine (DPPC), 1,2-distearoyl-sn-glycero-3-phosphocholine (DSPC), 1,2-distearoyl-sn-glycero-3-phosphoethanolamine-N-[poly(ethylene glycol)-2000] (DSPE-PEG(2000)), and 1,2-distearoyl-sn-glycero-3-phosphoethanolamine-N-[PDP-poly(ethylene glycol)-2000] (DSPE-PEG(2000)-PDP) for liposome preparation were purchased from Avanti Polar Lipids (Alabaster, Ala.). Fluorescently tagged lipid, Lissamine rhodamine B 1,2-dihexadecanoyl-sn-glycero-3-phosphoethanolamine (RhodaminePE), was purchased from Life Technologies (Grand Island, N.Y., USA). Size exclusion chromatography used CL-4B Sepharose gel, purchased from Sigma-Aldrich (St. Louis, Mo., USA). Human serum with complement C3, and human serum depleted of complement C3 were obtained from Quidel Corporation (Athens, Ohio, USA). Flow cytometry antibodies, PE/Dazzle 594 anti-human CD3, PerCP/Cy5.5 anti-human HLA-DR, APC anti-human CD14, Alexa Fluor 700 anti-human CD11c, APC/Cy7 anti-human CD11b, Pacific Blue anti-human CD15, Brilliant Violet 650 anti-human CD20, Brilliant Violet 605 anti-human CD33, Brilliant Violet 785 anti-human CD56, FITC anti-mouse CD45, PE anti-mouse CD25, PE/Dazzle 594 anti-mouse CD19, PerCP anti-mouse Ly-6G, PE/Cy7 anti-mouse CD11c, APC anti-mouse CD3, Alexa Fluor 700 anti-mouse CD11b, APC/Cy7 anti-mouse CD8b, Brilliant Violet 421 anti-mouse FOXP3, Brilliant Violet 510 anti-mouse Ly-6C, Brilliant Violet 605 anti-mouse IA/IE, Brilliant Violet 650 anti-mouse F4/80, Brilliant Violet 785 anti-mouse CD4, were purchased from BioLegend (San Diego, Calif., USA). Flow cytometry antibody, PC7 anti-human CD45, was purchased from Beckman Coulter (Brea, Calif., USA). All other chemicals, reagents, and kits were purchased from Thermo Fisher Scientific (Pittsburgh, Pa., USA).

ii. Cell Lines

The A20-OVA cell line was kindly provided by Dr. Gang Zhou (Augusta University, Atlanta, Ga.). This cell line is a lymphoma tumor cell line that has been stably transfected with ovalbumin as a mock tumor antigen. Tumor cells were cultured in complete medium (RPMI, 10% heat inactivated FBS, 1% penicillin/streptomycin, 0.05 mM 2-mercaptoethanol) and incubated at 37° C. in 5% CO2. The reporter T cell line, I-Ad-restricted OVA-specific T cell hybridoma D011, a generous gift from Dr. David Underhill (UCLA, Los Angeles, Calif.), is activated only by APCs presenting OVA peptides and expresses GFP when activated. Hybridoma T cells were cultured in complete medium (RPMI, 10% heat inactivated FBS, 1% penicillin/streptomycin, 0.05 mM 2-mercaptoethanol) and incubated at 37° C. in 5% CO2.

iii. Liposome Preparation

Liposomes were prepared using a previously described film hydration method. OPSS-liposomes are formulated using DSPE-PEG(2000)-PDP; control-liposomes are formulated using DSPE-PEG(2000). OPSS liposomes were made by mixing DPPC/DSPC/DSPE-PEG(2000)-PDP/DSPE-PEG(2000)/RhodaminePE in chloroform at a molecular ratio of 83:12:1:3:1. Control-liposomes were made following the same procedure and maintaining the same ratio, substituting DSPE-PEG(2000)-PDP with DSPE-PEG(2000). Lipid mixtures were dried under nitrogen stream for 1 hour to remove chloroform and the resulting lipid film was rehydrated with 0.7 mL of filtered water for non-protein encapsulated liposomes. Liposomes containing ovalbumin (OVA) were rehydrated with 0.7 mL 80 mg/mL ovalbumin solution and liposomes containing fluorescent DQ-OVA were rehydrated in 0.7 mL of 1 mg/mL DQ-OVA solution. Liposomes were then extruded 9 times through a 400 nm polycarbonate membrane filter at 47° C. Extruded liposomes were column purified using a CL-4B sepharose column hydrated in 1× PBS, pH 7.4. The concentration of control- and OPSS-liposome samples were normalized using a NanoDrop 2000 UV-Vis spectrophotometer, observing the rhodamine peak and diluting to a lipid concentration of 0.875 mg lipid/mL. Liposome size was determined using a Malvern Zetasizer Nano-S (Malvern Instruments, Malvern, UK); control-liposomes were measured as 262.1±65.74 nm, and OPSS-liposomes were measured as 265.4±101.6 nm. Encapsulation efficiency of OVA was determined by encapsulation of Alexa Fluor 488-OVA (1 mg/ml) and OVA (79 mg/ml) for a final OVA concentration of 80 mg/ml. After column purification, rhodamine fluorescence was used to determine liposomal concentration in the peak collected fraction and Alexa Fluor 488 fluorescence intensity was used to determine the level of OVA encapsulation. Encapsulation efficiency was estimated at 5.5%. The peak fraction of collected liposomes had a 1:240 dilution of OVA compared to the rehydration solution, and this dilution was used to match control levels of non-encapsulated OVA in experimentation.

iv. In Vitro Analysis of Antigen Processing and Presentation

Human whole blood, obtained from healthy volunteers, was collected in heparinized tubes. The blood draw protocol was approved by the UAA Institutional Review Board, in accordance with the U.S. Department of Health and Human Services requirements for the protection of human research subjects (45 CFR 46 as amended/revised), and all volunteer donors provided written informed consent. Peripheral blood mononuclear cells (PBMC) were isolated from whole blood using Ficoll-paque gradient separation. Isolated PBMCs were re-suspended in serum-free RPMI and plated at 1.6×10⁵ cells per well in a 96-well V-bottom plate.

For fluorescence microscopy, monocytes were isolated from PBMC to enrich for antigen presenting cells that take up liposomes. Monocyte isolation was performed by negative selection using a monocyte enrichment kit (Becton Dickinson, San Jose, Calif., USA).

Antigen processing by cells was analyzed using DQ-OVA (Molecular Probes), which fluoresces green after proteolytic degradation. 10 μL of rhodamine labeled OPSS- and control-liposomes, containing DQ-OVA, were incubated in 10 μL of C3-positive and -negative serum for 1 hour prior to addition to either PBMC or enriched monocytes. Liposomes and serum were added to cells (for a final serum concentration of 10%) and incubated for 3 hours at 37° C., 5% CO₂. Cells were centrifuged at 500×g for 5 minutes and rinsed twice in 1× PBS. Cells were analyzed by fluorescence microscopy and flow cytometry for both liposome internalization (rhodamine) and antigen processing and presentation (DQ-OVA).

v. Fluorescence Microscopy

For fluorescence microscopy, cells were transferred to a V-bottom plate, centrifuged 500×g 5 minutes, and rinsed twice with 1× PBS before transfer to a Falcon flat-bottom microtest 96-well assay plate, black/clear bottom (Becton Dickinson Labware, Franklin Lakes, N.J., USA) for imaging. Photos were taken using a Leica DMI6000B inverted fluorescence microscope (Leica Microsystems, Buffalo Grove, Ill., USA) and a 10× objective utilizing Leica Application Suite, version 3.7.0 software (Leica microsystems Inc., Wetzlar, Germany).

vi. Flow Cytometry Analysis

Samples were analyzed by flow cytometry to determine cell types, liposome uptake, and to quantify fluorescence. Cellular internalization of rhodamine labeled liposomes was determined by mean fluorescence intensity of rhodamine, detected on the PE channel. Antigen processing and presentation of DQ-OVA were determined by mean fluorescence intensity, detected on the FITC channel. Cell types were determined by fluorescence of specific cell marker antibodies, and cell type selection method was followed as previously described. Staining was performed on 1.6×10⁵ cells in a 96-well V-bottom plate and incubated with a master mix containing FACS buffer (1× PBS, 1% BSA) and 1 μL of each selected antibody for 20 minutes in the dark at 4° C. Cells were then centrifuged in a Sorvall T6000D centrifuge, re-suspended in 200 μL of FACS buffer, and analyzed using a Beckman Coulter CytoFLEX flow cytometer with CytExpert software (Beckman Coulter, Brea, Calif., USA).

vii. Generation of Bone Marrow Derived Dendritic Cells

Bone marrow was extracted from adult BALB/c mice. Mice were euthanized and femur and humerus bones removed and cleaned of tissue. Bones were kept in RPMI in a sterile petri dish, and bone marrow was flushed from bones using RPMI and a 1 mL insulin syringe. Extracted bone marrow was filtered through a 100 μm nylon mesh filter and rinsed twice in RPMI. Bone marrow cells were then counted and plated at a density of 2×10⁶ cells in culture medium (RPMI, 10% heat inactivated FBS, 1% penicillin/streptomycin). Granulocyte-macrophage colony stimulating factor (GM-CSF) and Interleukin-4 (IL-4) were added to the culture medium at 40 ng/mL and 20 ng/mL, respectively. Cells were incubated at 37° C. and medium with GM-CSF and IL-4 was replenished after 3 days. On day 6, non-adherent and loosely-adherent cells were harvested by pipetting and re-suspended in culture media.

viii. T Cell Activation

10 μL of OPSS-liposomes containing OVA, control-liposomes containing OVA and free OVA matching the amount encapsulated in OPSS-liposomes (see liposome preparation) were incubated in 10 μL C3-positive and -negative serum for 1 hour at room temperature prior to addition to 80 μL of bone marrow-derived dendritic cells in RPMI (1% penicillin/streptomycin) containing 1.6×105 cells per well in a U-bottom plate. Final serum concentration was therefore 10%. Cells were incubated with liposomes or controls for 24 hours at 37° C., 5% CO₂. Cells were rinsed twice in RPMI to remove any residual, non-internalized liposomes, and re-suspended in culture medium (RPMI, 10% serum, 1% penicillin/streptomycin). A reporter T cell line, I-Ad-restricted OVA-specific T cell hybridoma D011, was added to the dendritic cells at a 1:1 ratio and incubated for 24 hours. These reporter T cells are activated only by APCs presenting OVA peptides and expresses GFP when activated. The co-cultures were then analyzed for T cell activation (GFP expression) by fluorescence microscopy and flow cytometry.

ix. A20-OVA Tumor Inoculation and Mouse Model

Female and male 6-10 week old BALB/c mice were obtained from The Jackson Laboratory (Sacramento, Calif., USA). Mice were housed in the University of Alaska Anchorage (UAA) vivarium, and all experiments were approved by the UAA Institutional Animal Care and Use Committee. A20-OVA cells grown in complete medium were rinsed twice in 1× PBS. Mice were shaved and kept under anesthesia using isoflurane. Mice received subcutaneous injections in their left and right flanks, 1.5×10⁶ A20-OVA cells in 25 μl of PBS per injection. Treatments were started when tumors became palpable (approximately 10-14 days). Mice were separated into groups of 3 (2 females and 1 male per group), and received local subcutaneous injections of 100 μL of 1× PBS, non-encapsulated OVA equal to the encapsulated amount (1:240 dilution of 80 mg/mL OVA in 1× PBS) based on encapsulation efficiency measurements (see liposome preparation), control-liposomes, or OPSS-liposomes. Both control- and OPSS-liposomes were rehydrated in 80 mg/mL OVA. Mice only received injections on one side; the opposite side was measured to document systemic response to treatment. Mice received 7 total injections, on days 1, 2, 4, 6, 8, 10, and 12 (day 1 being the first injection). Tumor measurements were made before all injections and continuing for 4 days after injections were complete, using a digital caliper. Volumes were reported as mm3: [(4/3)π(length*width*minimum)/8]. Mice were monitored daily for signs of discomfort and distress, and were euthanized in accordance with IACUC approved standards.

x. Analysis of Mouse Blood

Mice were euthanized following therapy, 4 days after the last injection and blood was collected via cardiac puncture and placed in heparinized tubes. Plasma was separated from blood via centrifugation at 500×g for 15 minutes and frozen at −80° C. for subsequent analysis. Red blood cell lysis buffer (eBioscience) was added to the remaining blood for 10 minutes at room temperature. Samples were then prepared for flow cytometry analysis or frozen in culture media supplemented with 10% dimethyl sulfoxide for later use.

xi. Liver Toxicity Assays

Mouse plasma levels of aspartate transaminase (AST) and alanine transaminase (ALT) were determined using kits purchased from BioAssay Systems (Hayward, Calif., USA). Plasma was collected from mice as previously described. Plasma samples were diluted 1:1 in assay buffer prior to addition to a Falcon flat-bottom microtest 96-well assay plate, black/clear bottom (Becton Dickinson Labware, Franklin Lakes, N.J., USA). Absorbance was measured at 340 nm at 5 and 10 minutes using a BioTek Synergy HT plate reader, and enzyme activity was calculated according to the protocol.

xii. ELISA

Mouse plasma levels of anti-OVA IgG1 were determined by ELISA using a kit purchased from Cayman Chemicals (Ann Arbor, Mich., USA). Blood was collected in heparinized tubes from mice via cardiac puncture 4 days after the last injection. Plasma was collected from blood via centrifugation at 500×g for 15 minutes. Plasma samples were diluted 1:2000 in assay buffer prior to assay, and the provided kit procedure was followed. Absorbance was measured at 450 nm using a BioTek Synergy HT plate reader utilizing GenS software, version 2.01.

xiii. Statistical Analysis

Data is presented as mean +/−standard error (n=3). The Mann-Whitney-U test was used for studies involving mice, due to the small sample size and non-normal distribution. P values of less than 0.05 were considered significant.

2. Results

i. APCs Internalize C3-Liposomes and Process Antigen

Liposomes that contain an OPSS group have the ability to form a disulfide bond with activated complement C3 proteins, leading to uptake by antigen presenting cells through their complement receptor. To determine if liposomes could deliver antigen and enhance antigen presentation, liposomes were formulated to contain an encapsulated antigen, DQ-OVA. APC uptake of DQ-OVA loaded liposomes was determined via a fluorescent rhodamine label incorporated into the membranes of both OPSS- and control-liposomes. Antigen processing of DQ-OVA was observed through production of FITC fluorescence that occurs as DQ-OVA undergoes proteolytic degradation in the endosome for antigen presentation.

OPSS liposomes were incubated in human serum, containing complement C3 proteins, to produce targeted C3-bound liposomes (C3-liposomes). Liposomes lacking the OPSS group (control-liposomes) were incubated in human serum simultaneously; these liposomes do not form bonds with complement C3, creating a control, non-targeted liposome. Additional controls included both OPSS- and control-liposomes incubated in human serum depleted of complement C3 protein and non-encapsulated free DQ-OVA administered at the same concentration as encapsulated in liposomes. Serum-incubated liposomes were administered to Ficoll-isolated white blood cells from whole human blood.

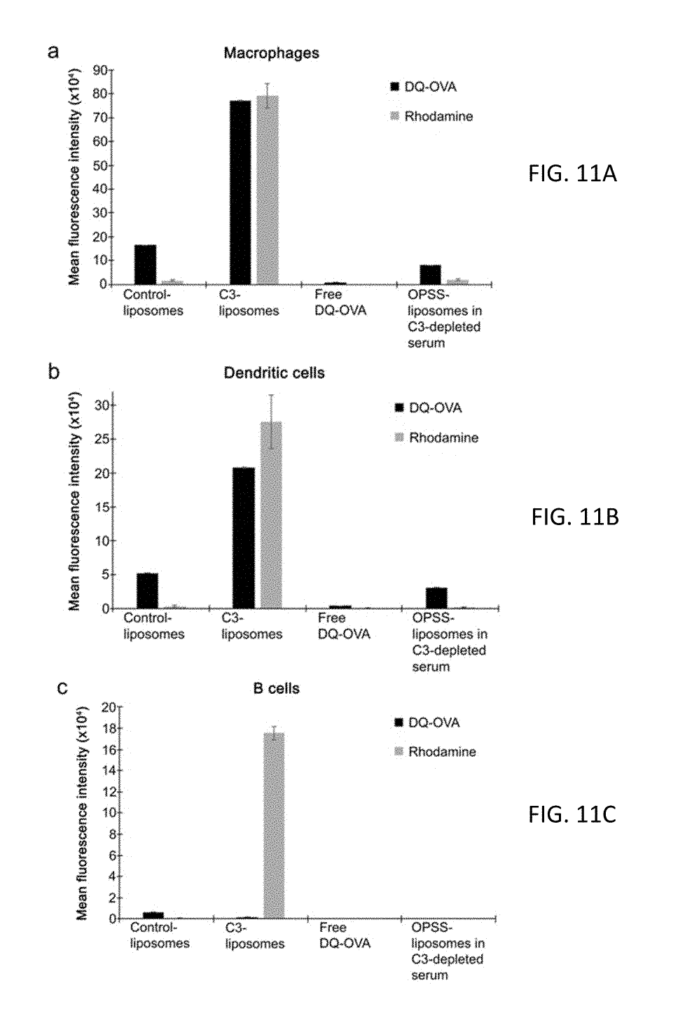

Flow cytometry analysis of rhodamine fluorescence revealed extensive uptake of C3-liposomes by the three antigen presenting cell types, macrophages (CD11b+CD14+), dendritic cells(CD11c+) and B cells(CD20+) (FIG. 11), confirming previously reported results. 19 In addition, DCs and macrophages showed intense FITC fluorescence, indicating proteolysis of delivered DQ-OVA (FIGS. 11a and 11b). Interestingly, B cells bound high levels of C3-liposomes but did not show evidence of antigen processing (FIG. 11c). The high level of rhodamine and FITC in APCs was only observed when DQ-OVA antigen was delivered by C3-liposomes incubated in complete human serum. Control-liposomes without the OPSS-group and liposomes incubated in C3-depleted serum exhibited relatively low uptake by APCs. Based on these results showing the necessity of both complement C3 in serum and the OPSS group on liposomes, uptake of liposomes is attributed to complement C3 proteins bound to the liposomal OPSS-groups, which targets complement receptors on the plasma membranes of APCs.

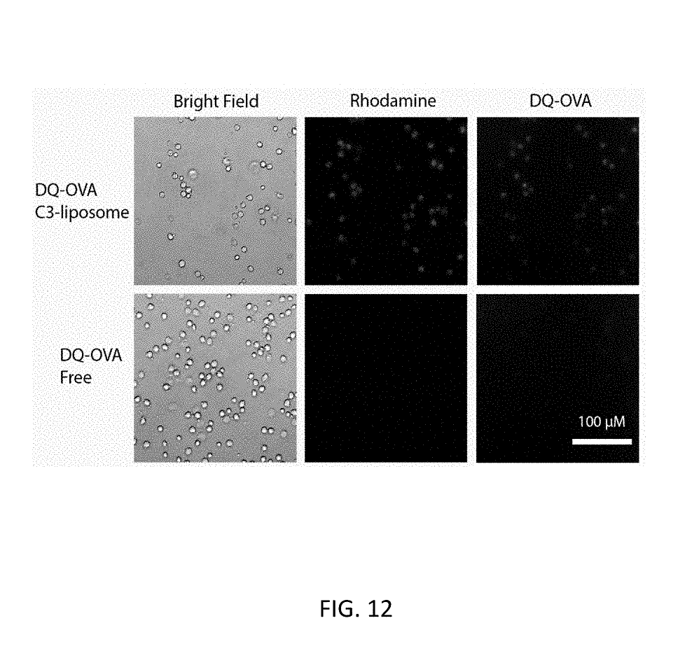

Importantly, based on FITC intensity, uptake of DQ-OVA C3-liposomes resulted in a 91-fold increase in processed DQ-OVA in macrophages and a 54-fold increase in dendritic cells, when compared to non-targeted free DQ-OVA at the same concentration (FIG. 11). This increase in antigen delivery is confirmed by fluorescent microscopy, which shows isolated human monocytes exposed to either rhodamine labeled C3-liposomes that contain DQ-OVA or free DQ-OVA at the same concentration (FIG. 12). The intense red rhodamine fluorescence shows the high uptake of liposomes by monocytes and the DQ-OVA antigen processing shown in green is remarkably higher when targeted within C3-liposomes.

ii. C3-Liposome Antigen Delivery to APCs Leads to T Cell Activation

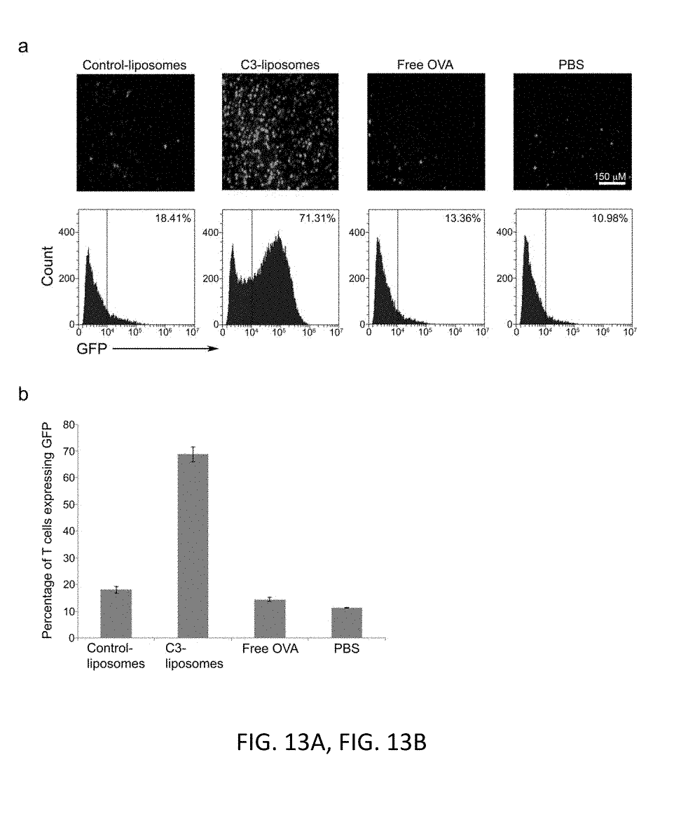

To evaluate the ability of C3-liposomes to deliver OVA antigen to APCs and activate an antigen specific T cell response, OVA was delivered to bone marrow-derived dendritic cells (BMDCs), and then co-incubated with the OVA-specific reporter T cell line D011. The T cell receptor on D011 T cells recognizes antigenic epitopes of OVA peptides when they are presented by APCs. In response to APC presentation of OVA epitopes and stimulation of the T cell receptor, GFP is expressed at a high level within the D011 T cells, allowing for a direct measurement of OVA specific T cell activation. GFP fluorescence was observed by means of both fluorescence microscopy and flow cytometry (FIG. 13).

Co-cultures with OVA encapsulated C3-liposomes resulted in the highest percentage of T cell activation (68.7±2.7%) (FIG. 3). Controls, including cultures treated with the equivalent non-encapsulated free OVA displayed significantly lower levels of T cells activation, (control-liposomes: 18.0±1.3%; free OVA: 14.4±0.8%; PBS: 11.3±0.1%). These results confirm that C3-bound liposomes improve antigen (OVA) presentation by APCs and that OVA epitopes are specifically recognized by T cells resulting in T cell activation.

iii. OVA C3-Liposome Induced Antigen Specific Immune Response Eliminates Tumors in Mice

To evaluate if C3-liposomes could deliver tumor antigen and activate tumor antigen specific immune response in vivo, OVA C3-liposomes were used to treat A20-OVA lymphoma tumors in male and female BALB/c mice. A20-OVA cells have been transfected with OVA as a mock tumor antigen and can be used to determine if OVA vaccination leads to reduction in tumor growth. Mice were injected with A20-OVA cells on each flank to establish tumors. Once tumors were palpable (100 mm³), each mouse received a local subcutaneous injection of a specific treatment at only one tumor site while the other tumor was left untreated in order to gauge the systemic response to therapy.

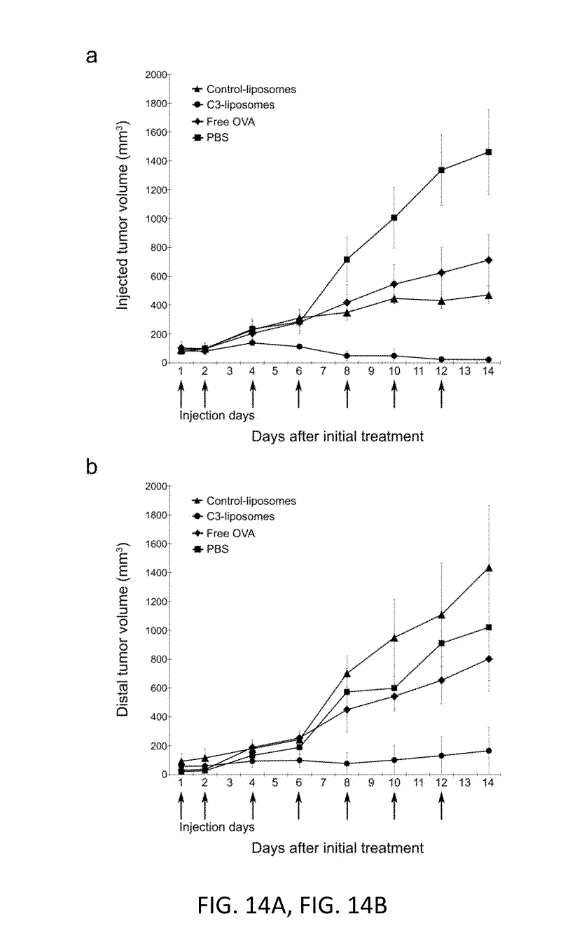

Once tumors became palpable, mice were split evenly into four groups to normalize average tumor sizes. Groups were randomly selected to receive PBS, C3-liposomes, control-liposomes, or non-encapsulated free OVA injections. Liposomes and non-encapsulated OVA contained equivalent amounts of OVA. The treatment schedule consisted of two consecutive intratumoral injections, followed by 5 every other day intratumoral injections, for a total of 7 injections. Mice receiving OVA C3-liposome treatments had reduced tumor growth of the injected tumors by the third injection, reduced growth of the distal tumors by the fourth injection, and complete elimination of tumors by day six in two out of three mice (FIG. 14). Importantly the elimination of tumors in 2 out of 3 mice and the reduction of tumor size in the third mouse, occurred at both the injected and distal tumor, indicating an effective systemic anti-tumor immunity in response to antigen delivery with C3-liposomes. All other treatment groups exhibited continuous tumor growth, with some response on the injected side (FIG. 14a), but no significant response in the distal tumor (FIG. 14b).

To assess treatment toxicity, liver enzymes aspartate transaminase (AST) and alanine transaminase (ALT) were measured in mouse plasma, since elevated levels of AST and/or ALT in blood are indicators of liver damage. Results from the 4 different treatment groups fell within normal ranges, with no significant differences between treatment groups (ALT, control-liposomes: 71.7±23.8 U/L, C3-liposomes: 60.3±19.5 U/L, free OVA: 51.2±34.7 U/L, PBS: 40.6±26.4 U/L, normal range: 15-84 U/L; AST, control-liposomes: 88.4±40.9 U/L, C3-liposomes: 171.0±42.6 U/L, free-OVA: 110.4±25.6 U/L, PBS: 82.6±18.5 U/L, normal range: 54-298 U/L).

iv. OVA C3-Liposomes Decrease MDSCs and Increase Circulating B Cells

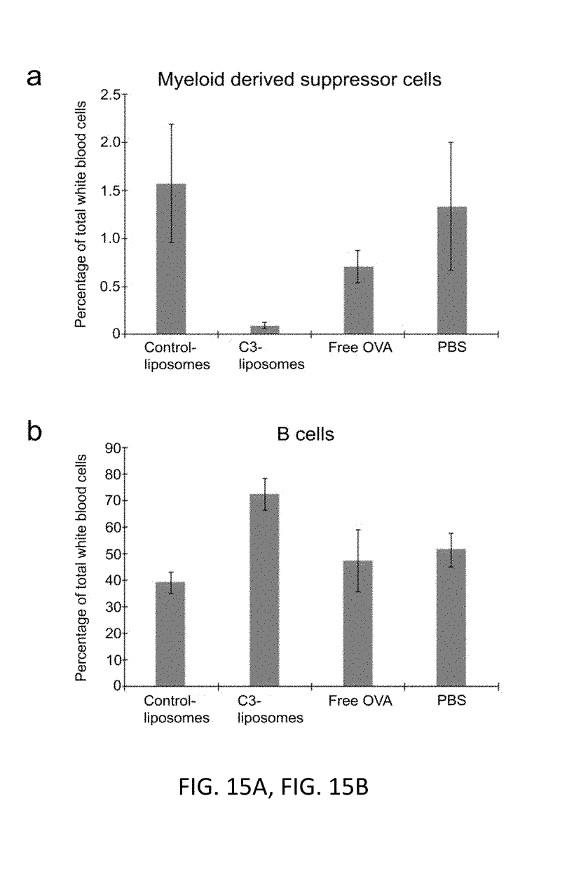

Two weeks following initial treatment, mice were euthanized and blood was collected for analysis of circulating immune cells. OVA C3-liposome treated mice had significantly lower levels of systemic CD11b+Ly6c^{hi} myeloid derived suppressor cells (MDSCs), compared to mice treated with non-OPSS OVA control-liposomes, free OVA or PBS (FIG. 15a). Additionally, OVA C3-liposome treated mice had elevated percentages of CD20+ B cells, compared to all other treatment groups (FIG. 15b). No significant differences in blood T cell numbers were found between treatment groups (data not shown), but this can be due to the length of time between tumor reduction and analysis of white blood cell numbers. T cell infiltration of tumors could not be evaluated since two out of three mice were tumor free at the end of the study.

v. OVA Treatments Increase Anti-OVA IgG1

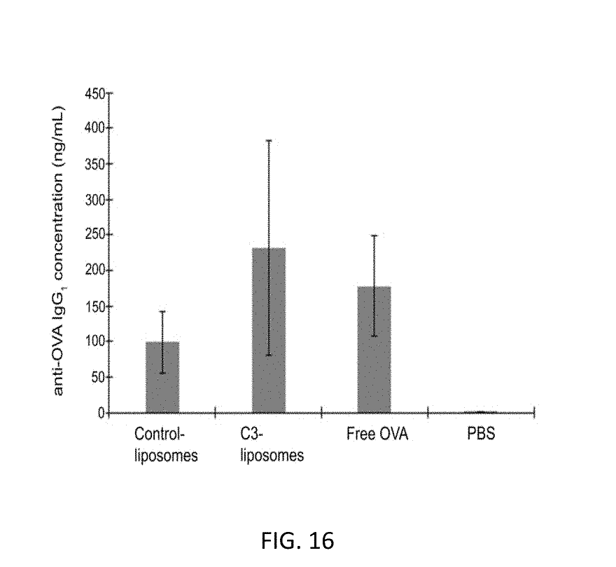

Plasma was collected from mouse blood samples to determine the levels of circulating anti-OVA IgG1 between treatment groups. ELISA analysis of plasma samples revealed significant increases in anti-OVA IgG1 in all treatment groups compared to the PBS-treated mice (FIG. 16). These results indicated mice exposed to OVA antigen produced a humoral immune response to OVA within 14 days following the first injection.

3. Discussion

Antigen presenting cells initiate an immune response by processing antigens and presenting antigenic epitopes to T cells. Tumor vaccines aim to deliver tumor antigens to APCs to bolster antigen presentation and thereby enhance the immune response against cancer. With the progression of patient tumor sequencing, there is a growing library of identified tumor antigens and an increasing need for technologies that can deliver tumor antigens directly to APCs. The goal of this research is to continue development of complement-bound C3-liposomes that target APCs through complement-mediated pathways, with the hopes of improving T cell recognition of encapsulated tumor antigens.

C3-liposomes are lipid particles, approximately 260 nm in size, which bind to activated complement C3 proteins by virtue of a lipid-attached OPSS group. These liposomes resemble a complement coated pathogen and are targeted for phagocytosis by cells with receptors for activated complement C3 fragments. Complement C3 is the most abundant protein in the complement system and is activated by cleavage of a reactive thioester into C3a and C3b, the latter of which acts as an opsonizing agent. C3b fragments are further metabolized to C3c and C3d/C3dg. Complement Receptor 3 (CD11b/CD18), the most common type of complement receptor on monocytes and polymorphonuclear cells, binds iC3b fragments and is located on macrophages, dendritic cells, neutrophils and MDSCs. Complement Receptor 2 (CD21) is found on B cells and binds iC3b, C3d, and C3dg fragments. Many of these activated C3 fragments bind covalently and specifically to C3-liposomes. These components of complement C3 allow for uptake of C3-liposomes into all three types of APCs: dendritic cells, macrophages and B cells.

C3-liposomes that encapsulate tumor antigen are taken up by all three APCs, resulting in efficient antigen delivery and processing in macrophages and dendritic cells. Compared to non-targeted antigen, C3-liposomes greatly improve uptake and proteolytic cleavage of encapsulated antigen, which is the first step in initiating an adaptive immune response. Interestingly, B cells take up high levels of C3-liposomes but do not process the encapsulated antigen. Further experimentation will be needed to determine why antigen is not processed in B cell endosomes or if this is an artifact of the in vitro system. BMDCs targeted by C3-liposomes loaded with ovalbumin as antigen activate T cells that display the T cell receptor for the delivered antigen. Again, this activation is dependent on liposome encapsulation of antigen and delivery mediated by C3 targeting. Taken together, these in vitro results show the ability of C3-liposomes to enhance antigen delivery and subsequent T cell activation.

Treatment with C3-liposomes containing tumor antigen at a single tumor site leads to a systemic anti-tumor immune response that eliminates tumors in a majority of treated mice. Compared to non-targeted tumor antigen, antigen delivery with C3-liposomes leads to superior growth reduction in tumors that were injected intratumorally. To evaluate if there was a systemic immune response, tumor growth was measured at a distal site that did not receive direct injections. C3-liposome delivery of antigen is the only treatment that results in a systemic response, leading to tumor reduction in all three mice and complete elimination of injected and distal tumors in two out of three mice. Surgical subcutaneous analysis of tumor free mice revealed no evidence of tumor lesions, angiogenic vessels and skin appeared healthy in all regards.

Treatment with C3-liposomes results in a significant reduction in number of MDSCs and an increase in circulating B cells. MDSCs are a heterogeneous population of immature cells that expand in number in response to signals and cytokines released from the tumor. Previously, C3-liposomes were shown to be taken up by MDSCs which display complement receptor 3. The decrease in MDSCs may possibly be due to a reduction in overall tumor burden in mice treated with C3-liposomes, but may also be due to elimination or reprogramming of MDSCs to a differentiated phenotype in response to the binding and internalization of C3-liposomes. In cancer patients, MDSC are a key cell type responsible for promoting immunosuppression, with elevated systemic levels correlated with cancer progression and poor prognosis. If C3-liposomes can reverse MDSCs immune suppression, they could provide an important mechanism for improving immunotherapy.

Even with successful antigen delivery to APCs and subsequent T cell activation by C3-liposomes, T regulatory cells, MDSCs and tumor cell expression of PD-L1 could limit the effectiveness of an immune response. Therefore, C3-liposomes will be most effective if used in combination with existing immunotherapies. Two antibody-based cancer immunotherapies, anti-PD-1 and anti-CTLA-4, have had success in treating melanoma, among other cancers. CTLA-4 is a receptor located on T regulatory cells and is responsible for blocking the interaction between APCs and T cells. PD-1 is a receptor located on T cells that results in T cell anergy or apoptosis when bound by its ligand (PD-L1), which is commonly upregulated by tumor cells. The combination of increased antigen presentation and reduced number of MDSCs resulting from C3-liposome treatment along with reduction of T regulatory cells by anti-CTLA-4 treatment and decreased T cell anergy due to anti-PD-1 treatment could result in a powerful anti-tumor immune response.

Immunotherapies are often limited by autoimmunity and other toxicities associated with the treatment. C3-liposomes are composed of neutral lipids and have a polyethylene glycol layer that reduces aggregation and results in minimal toxicity, as revealed by normal AST and ALT liver enzymatic levels and by no evidence of pulmonary distress after treatment. C3-liposomes bind to endogenous complement C3 in the blood, which should negate unwanted immunogenicity due to foreign targeting ligands. C3-liposomes use a small molecule for binding complement, which could provide a cost-efficient means of treatment, without the need for labor intensive ex vivo cultures, expensive patient-specific reagents, or immunoglobulin-based targeting.

The results described here demonstrate the potential of C3-liposomes to improve antigen delivery and T cell activation. Further in vivo experimentation with C3-liposomes will focus on delivering tumor antigens derived from spontaneous mutations in mouse tumor cell lines and on testing C3-liposome treatment in combination with anti-CTLA-4 and anti-PD-1 immunotherapies. With a growing library of known tumor antigens, C3-liposomes could provide an important technology for enhancing cancer immunotherapy.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Cerezo-Wallis D, Soengas M. Understanding Tumor-Antigen Presentation in the New Era of Cancer Immunotherapy. Curr Pharm Des. 2016.
2. Markowitz J, Wesolowski R, Papenfuss T, Brooks T R, Carson W E, 3rd. Myeloid-derived suppressor cells in breast cancer. Breast Cancer Res Treat. 2013; 140(1):13-21.
3. Tagliamonte M, Petrizzo A, Tornesello M L, Buonaguro F M, Buonaguro L. Antigen-specific vaccines for cancer treatment. Hum Vaccin Immunother. 2014; 10(11):3332-3346.
4. Nefedova Y, Fishman M, Sherman S, Wang X, Beg A A, Gabrilovich D I. Mechanism of all-trans retinoic acid effect on tumor-associated myeloid-derived suppressor cells. Cancer Res. 2007; 67(22):11021-11028.
5. Youn J I, Nagaraj S, Collazo M, Gabrilovich D I. Subsets of myeloid-derived suppressor cells in tumor-bearing mice. J Immunol. 2008; 181(8):5791-5802.
6. Alcorlo M, Martinez-Barricarte R, Fernandez F J, et al. Unique structure of iC3b resolved at a resolution of 24 A by 3D-electron microscopy. Proc Natl Acad Sci USA. 2011; 108(32):13236-13240.
7. Kullberg M, Martinson H, Mann K, Anchordoquy T J. Complement C3 mediated targeting of liposomes to granulocytic myeloid derived suppressor cells. Nanomedicine. 2015.
8. Fan Y, Moon J J. Nanoparticle Drug Delivery Systems Designed to Improve Cancer Vaccines and Immunotherapy. Vaccines (Basel). 2015; 3(3):662-685.
9. Joshi M D, Unger W J, Storm G, van Kooyk Y, Mastrobattista E. Targeting tumor antigens to dendritic cells using particulate carriers. J Control Release. 2012; 161(1):25-37.
10. Irache J M, Salman H H, Gamazo C, Espuelas S. Mannose-targeted systems for the delivery of therapeutics. Expert Opin Drug Deliv. 2008; 5(6):703-724.
11. Xu L, Anchordoquy T. Drug delivery trends in clinical trials and translational medicine: challenges and opportunities in the delivery of nucleic acid-based therapeutics. J Pharm Sci. 2011; 100(1):38-52.
12. Merle N S, Church S E, Fremeaux-Bacchi V, Roumenina L T. Complement System Part I—Molecular Mechanisms of Activation and Regulation. Front Immunol. 2015; 6:262.
13. Merle N S, Noe R, Halbwachs-Mecarelli L, Fremeaux-Bacchi V, Roumenina L T. Complement System Part II: Role in Immunity. Front Immunol. 2015; 6:257.
14. Kullberg M, Mann K, Anchordoquy T J. Targeting Her-2+ breast cancer cells with bleomycin immunoliposomes linked to LLO. Mol Pharm. 2012; 9(7):2000-2008.
15. Hess M W, Schwendinger M G, Eskelinen E L, et al. Tracing uptake of C3dg-conjugated antigen into B cells via complement receptor type 2 (CR2, CD21). Blood. 2000; 95(8):2617-2623.
16. Geyer M B, Brentjens R J. Review: Current clinical applications of chimeric antigen receptor (CAR) modified T cells. Cytotherapy. 2016.
17. Soliman H. Immunotherapy strategies in the treatment of breast cancer. Cancer Control. 2013; 20(1):17-21.
18. Almand B, Resser J R, Lindman B, et al. Clinical significance of defective dendritic cell differentiation in cancer. Clin Cancer Res. 2000; 6(5):1755-1766.
19. Diaz-Montero C M, Salem M L, Nishimura M I, Garrett-Mayer E, Cole D J, Montero A J. Increased circulating myeloid-derived suppressor cells correlate with clinical cancer stage, metastatic tumor burden, and doxorubicin-cyclophosphamide chemotherapy. Cancer Immunol Immunother. 2009; 58(1):49-59.
20. Kusmartsev S, Cheng F D, Yu B, et al. All-trans-retinoic acid eliminates immature myeloid cells from tumor-bearing mice and improves the effect of vaccination. Cancer Research. 2003; 63(15):4441-4449.
21. Mirza N, Fishman M, Fricke I, et al. All-trans-retinoic acid improves differentiation of myeloid cells and immune response in cancer patients. Cancer Res. 2006; 66(18):9299-9307.
22. Ugel S, Delpozzo F, Desantis G, et al. Therapeutic targeting of myeloid-derived suppressor cells. Curr Opin Pharmacol. 2009; 9(4):470-481.
23. Candolfi M, Curtin J F, Yagiz K, et al. B cells are critical to T-cell-mediated antitumor immunity induced by a combined immune-stimulatory/conditionally cytotoxic therapy for glioblastoma. Neoplasia. 2011; 13(10):947-960.
24. Schultze J L, Michalak S, Seamon M J, et al. CD40-activated human B cells: an alternative source of highly efficient antigen presenting cells to generate autologous antigen-specific T cells for adoptive immunotherapy. J Clin Invest. 1997; 100(11):2757-2765.
25. Thomas S, Prendergast G C. Cancer Vaccines: A Brief Overview. Methods Mol Biol. 2016; 1403:755-761.
26. Busch D H, Frassle S P, Sommermeyer D, Buchholz V R, Riddell S R. Role of memory T cell subsets for adoptive immunotherapy. Semin Immunol. 2016; 28(1):28-34.
27. Xu Y, Dotti G. Selection bias: maintaining less-differentiated T cells for adoptive immunotherapy. J Clin Invest. 2016; 126(1):35-37.
28. Barrington R A, Schneider T J, Pitcher L A, et al. Uncoupling CD21 and CD19 of the B-cell coreceptor. Proc Natl Acad Sci USA. 2009; 106(34):14490-14495.
29. Lyubchenko T, Dal Porto J M, Holers V M, Cambier J C. Cutting edge: Complement (C3d)-linked antigens break B cell anergy. J Immunol. 2007; 179(5):2695-2699.
30. Tagliamonte M, Petrizzo A, Tornesello M L, Buonaguro F M, Buonaguro L. Antigen-specific vaccines for cancer treatment. Hum Vaccin Immunother. 2014; 10(11):3332-3346.
31. Cerezo-Wallis D, Soengas M. Understanding Tumor-Antigen Presentation in the New Era of Cancer Immunotherapy. Curr Pharm Des. 2016.
32. Geyer M B, Brentjens R J. Review: Current clinical applications of chimeric antigen receptor (CAR) modified T cells. Cytotherapy. 2016.
33. Thomas S, Prendergast G C. Cancer Vaccines: A Brief Overview. Methods Mol Biol. 2016; 1403:755-761.
34. Yang F, Jin H, Wang J, et al. Adoptive Cellular Therapy (ACT) for Cancer Treatment. Adv Exp Med Biol. 2016; 909:169-239.
35. Joshi M D, Unger W J, Storm G, van Kooyk Y, Mastrobattista E. Targeting tumor antigens to dendritic cells using particulate carriers. J Control Release. 2012; 161(1):25-37.
36. Kullberg M, Martinson H, Mann K, Anchordoquy T J. Complement C3 mediated targeting of liposomes to granulocytic myeloid derived suppressor cells. Nanomedicine. 2015.
37. Merle N S, Noe R, Halbwachs-Mecarelli L, Fremeaux-Bacchi V, Roumenina L T. Complement System Part II: Role in Immunity. Front Immunol. 2015; 6:257.
38. Alcorlo M, Martinez-Barricarte R, Fernandez F J, et al. Unique structure of iC3b resolved at a resolution of 24 A by 3D-electron microscopy. Proc Natl Acad Sci USA. 2011; 108(32):13236-13240.
39. Bevan M J. Cross-priming. Nat Immunol. 2006; 7(4):363-365.
40. Candolfi M, Curtin J F, Yagiz K, et al. B cells are critical to T-cell-mediated antitumor immunity induced by a combined immune-stimulatory/conditionally cytotoxic therapy for glioblastoma. Neoplasia. 2011; 13(10):947-960.
41. Schultze J L, Michalak S, Seamon M J, et al. CD40-activated human B cells: an alternative source of highly efficient antigen presenting cells to generate autologous antigen-specific T cells for adoptive immunotherapy. J Clin Invest. 1997; 100(11):2757-2765.
42. Hawiger D, Inaba K, Dorsett Y, et al. Dendritic cells induce peripheral T cell unresponsiveness under steady state conditions in vivo. J Exp Med. 2001; 194(6):769-779.
43. Bonifaz L, Bonnyay D, Mahnke K, Rivera M, Nussenzweig M C, Steinman R M. Efficient targeting of protein antigen to the dendritic cell receptor DEC-205 in the steady state leads to antigen presentation on major histocompatibility complex class I products and peripheral CD8+ T cell tolerance. J Exp Med. 2002; 196(12):1627-1638.
44. Lyubchenko T, Dal Porto J M, Holers V M, Cambier J C. Cutting edge: Complement (C3d)-linked antigens break B cell anergy. J Immunol. 2007; 179(5):2695-2699.
45. Hess M W, Schwendinger M G, Eskelinen E L, et al. Tracing uptake of C3dg-conjugated antigen into B cells via complement receptor type 2 (CR2, CD21). Blood. 2000; 95(8):2617-2623.
46. Barrington R A, Schneider T J, Pitcher L A, et al. Uncoupling CD21 and CD19 of the B-cell coreceptor. Proc Natl Acad Sci USA. 2009; 106(34):14490-14495.
47. Mahoney K M, Rennert P D, Freeman G J. Combination cancer immunotherapy and new immunomodulatory targets. Nat Rev Drug Discov. 2015; 14(8):561-584.
48. Fan Y, Moon J J. Nanoparticle Drug Delivery Systems Designed to Improve Cancer Vaccines and Immunotherapy. Vaccines (Basel). 2015; 3(3):662-685.
49. Busch D H, Frassle S P, Sommermeyer D, Buchholz V R, Riddell S R. Role of memory T cell subsets for adoptive immunotherapy. Semin Immunol. 2016; 28(1):28-34.
50. Xu Y, Dotti G. Selection bias: maintaining less-differentiated T cells for adoptive immunotherapy. J Clin Invest. 2016; 126(1):35-37.
51. Xu L, Anchordoquy T. Drug delivery trends in clinical trials and translational medicine: challenges and opportunities in the delivery of nucleic acid-based therapeutics. J Pharm Sci. 2011; 100(1):38-52.
52. Underhill D M, Bassetti M, Rudensky A, Aderem A. Dynamic interactions of macrophages with T cells during antigen presentation. J Exp Med. 1999; 190(12):1909-1914.
53. Sato M, Chamoto K, Nishimura T. A novel tumor-vaccine cell therapy using bone marrow-derived dendritic cell type 1 and antigen-specific Th1 cells. International immunology. 2003; 15(7):837-843.
54. Marabelle A, Kohrt H, Sagiv-Barfi I, et al. Depleting tumor-specific Tregs at a single site eradicates disseminated tumors. J Clin Invest. 2013; 123(6):2447-2463.
55. Chamoto K, Wakita D, Narita Y, et al. An essential role of antigen-presenting cell/T-helper type 1 cell-cell interactions in draining lymph node during complete eradication of class II-negative tumor tissue by T-helper type 1 cell therapy. Cancer Res. 2006; 66(3):1809-1817.
56. Sagiv-Barfi I, Kohrt H E, Czerwinski D K, Ng P P, Chang B Y, Levy R. Therapeutic antitumor immunity by checkpoint blockade is enhanced by ibrutinib, an inhibitor of both BTK and ITK. Proc Natl Acad Sci USA. 2015; 112(9):E966-972.
57. Ribas A, Butterfield L H, Hu B, et al. Generation of T-cell immunity to a murine melanoma using MART-1-engineered dendritic cells. J Immunother. 2000; 23(1):59-66.
58. Quaglino E, Mastini C, Forni G, Cavallo F. ErbB2 transgenic mice: a tool for investigation of the immune prevention and treatment of mammary carcinomas. Curr Protoc Immunol. 2008; Chapter 20:Unit 20 29 21-20 29-10.
59. Shirota H, Sano K, Hirasawa N, et al. B cells capturing antigen conjugated with CpG oligodeoxynucleotides induce Th1 cells by elaborating IL-12. J Immunol. 2002; 169(2):787-794.
60. Murad Y M, Clay T M. CpG oligodeoxynucleotides as TLR9 agonists: therapeutic applications in cancer. BioDrugs. 2009; 23(6):361-375.
61. Aubin E, Lemieux R, Bazin R. Indirect inhibition of in vivo and in vitro T-cell responses by intravenous immunoglobulins due to impaired antigen presentation. Blood. 2010; 115(9):1727-1734.
62. Leung S, Smith D, Myc A, Morry J, Baker J R, Jr. OT-II TCR transgenic mice fail to produce anti-ovalbumin antibodies upon vaccination. Cell Immunol. 2013; 282(2):79-84.
63. Draghiciu O, Lubbers J, Nijman H W, Daemen T. Myeloid derived suppressor cells-An overview of combat strategies to increase immunotherapy efficacy. Oncoimmunology. 2015; 4(1): e954829.
64. Khaled Y S, Ammori B J, Elkord E. Myeloid-derived suppressor cells in cancer: recent progress and prospects. Immunol Cell Biol. 2013; 91(8):493-502.
65. Coulie, P. G., Van den Eynde, B. J., van der Bruggen, P. & Boon, T. Tumour antigens recognized by T lymphocytes: at the core of cancer immunotherapy. Nat Rev Cancer 14, 135-146 (2014).
66. Bevan, M. J. Helping the CD8(+) T-cell response. Nat Rev Immunol 4, 595-602 (2004).
38. Tagliamonte, M., Petrizzo, A., Tornesello, M. L., Buonaguro, F. M. & Buonaguro, L. Antigen-specific vaccines for cancer treatment. Hum Vaccin Immunother 10, 3332-3346 (2014).
67. Gubin, M. M., Artyomov, M. N., Mardis, E. R. & Schreiber, R. D. Tumor neoantigens: building a framework for personalized cancer immunotherapy. J Clin Invest 125, 3413-3421 (2015).
68. Ilyas, S. & Yang, J. C. Landscape of Tumor Antigens in T Cell Immunotherapy. J Immunol 195, 5117-5122 (2015).
69. Cheng, W. F. et al. Fusion protein vaccines targeting two tumor antigens generate synergistic anti-tumor effects. PLoS One 8, e71216 (2013).
70. Dunn, G. P., Bruce, A. T., Ikeda, H., Old, L. J. & Schreiber, R. D. Cancer immunoediting: from immunosurveillance to tumor escape. Nat Immunol 3, 991-998 (2002).
71. Smyth, M. J., Dunn, G. P. & Schreiber, R. D. Cancer immunosurveillance and immunoediting: the roles of immunity in suppressing tumor development and shaping tumor immunogenicity. Adv Immunol 90, 1-50 (2006).
72. Schietinger, A. & Greenberg, P. D. Tolerance and exhaustion: defining mechanisms of T cell dysfunction. Trends Immunol 35, 51-60 (2014).
73. Nurieva, R., Wang, J. & Sahoo, A. T-cell tolerance in cancer. Immunotherapy 5, 513-531 (2013).
74. Miller, S. D., Turley, D. M. & Podojil, J. R. Antigen-specific tolerance strategies for the prevention and treatment of autoimmune disease. Nat Rev Immunol 7, 665-677 (2007).
75. Joshi, M. D., Unger, W. J., Storm, G., van Kooyk, Y. & Mastrobattista, E. Targeting tumor antigens to dendritic cells using particulate carriers. J Control Release 161, 25-37 (2012).
76. Fan, Y. & Moon, J. J. Nanoparticle Drug Delivery Systems Designed to Improve Cancer Vaccines and Immunotherapy. Vaccines (Basel) 3, 662-685 (2015).
77. Vudattu, N. K. et al. Humanized mice as a model for aberrant responses in human T cell immunotherapy. J Immunol 193, 587-596 (2014).
78. Tran Janco, J. M., Lamichhane, P., Karyampudi, L. & Knutson, K. L. Tumor-infiltrating dendritic cells in cancer pathogenesis. J Immunol 194, 2985-2991 (2015).
79. Palucka, K. & Banchereau, J. Cancer immunotherapy via dendritic cells. Nat Rev Cancer 12, 265-277 (2012).
80. Simerska, P. et al. Ovalbumin lipid core peptide vaccines and their CD4(+) and CD8(+) T cell responses. Vaccine 32, 4743-4750 (2014).
81. Irache, J. M., Salman, H. H., Gamazo, C. & Espuelas, S. Mannose-targeted systems for the delivery of therapeutics. Expert Opin Drug Deliv 5, 703-724 (2008).
82. Francian, A., Mann, K. & Kullberg, M. Complement C3-dependent uptake of targeted liposomes into human macrophages, B cells, dendritic cells, neutrophils, and MDSCs. Int J Nanomedicine 12, 5149-5161 (2017).
83. Kullberg, M., Martinson, H., Mann, K. & Anchordoquy, T. J. Complement C3 mediated targeting of liposomes to granulocytic myeloid derived suppressor cells. Nanomedicine 11, 1355-1363 (2015).
84. Merle, N. S., Church, S. E., Fremeaux-Bacchi, V. & Roumenina, L. T. Complement System Part I—Molecular Mechanisms of Activation and Regulation. Front Immunol 6, 262 (2015).
85. Merle, N. S., Noe, R., Halbwachs-Mecarelli, L., Fremeaux-Bacchi, V. & Roumenina, L. T. Complement System Part II: Role in Immunity. Front Immunol 6, 257 (2015).
86. Mayer, L. P., Dyer, C. A., Eastgard, R. L., Hoyer, P. B. & Banka, C. L. Atherosclerotic lesion development in a novel ovary-intact mouse model of perimenopause. Arterioscler Thromb Vasc Biol 25, 1910-1916 (2005).
87. Huang W C, S. J., Liou C J, et al. Enhancing Th1 cell activities in mice by short-term oral administration of Gynostemma pentaphyllum extracts. BioFormosa 42, 9-16 (2007).
88. Vigneron, N., Stroobant, V., Van den Eynde, B. J. & van der Bruggen, P. Database of T cell-defined human tumor antigens: the 2013 update. Cancer Immun 13, 15 (2013).
89. Lukacsi, S., Nagy-Balo, Z., Erdei, A., Sandor, N. & Bajtay, Z. The role of CR3 (CD11b/CD18) and CR4 (CD11c/CD18) in complement-mediated phagocytosis and podosome formation by human phagocytes. Immunol Lett 189, 64-72 (2017).
90. Sandor, N. et al. CR3 is the dominant phagocytotic complement receptor on human dendritic cells. Immunobiology 218, 652-663 (2013).
91. Carroll, M. C. & Isenman, D. E. Regulation of humoral immunity by complement. Immunity 37, 199-207 (2012).
92. Sun, H. et al. Increase in myeloid-derived suppressor cells (MDSCs) associated with minimal residual disease (MRD) detection in adult acute myeloid leukemia. Int J Hematol 102, 579-586 (2015).
93. Manjili, M. H., Wang, X. Y. & Abrams, S. Evolution of Our Understanding of Myeloid Regulatory Cells: From MDSCs to Mregs. Front Immunol 5, 303 (2014).
94. Marvel, D. & Gabrilovich, D. I. Myeloid-derived suppressor cells in the tumor microenvironment: expect the unexpected. J Clin Invest 125, 3356-3364 (2015).
95. Diaz-Montero, C. M. et al. Increased circulating myeloid-derived suppressor cells correlate with clinical cancer stage, metastatic tumor burden, and doxorubicin-cyclophosphamide chemotherapy. Cancer Immunol Immunother 58, 49-59 (2009).
96. Hodi, F. S. et al. Improved survival with ipilimumab in patients with metastatic melanoma. N Engl J Med 363, 711-723 (2010).
97. Pardoll, D. M. The blockade of immune checkpoints in cancer immunotherapy. Nat Rev Cancer 12, 252-264 (2012).
98. Krummel, M. F. & Allison, J. P. CD28 and CTLA-4 have opposing effects on the response of T cells to stimulation. J Exp Med 182, 459-465 (1995).
99. Sznol, M. & Chen, L. Antagonist antibodies to PD-1 and B7-H1 (PD-L1) in the treatment of advanced human cancer-response. Clin Cancer Res 19, 5542 (2013).

We claim:

1. A method of treating a disease or condition in a subject comprising:
administering to a subject a nanoparticle, wherein the nanoparticle comprises a therapeutic, wherein upon administration the nanoparticle binds to activated C3 present in the subject forming a nanoparticle-C3 conjugate, wherein the nanoparticle-C3 conjugate targets antigen presenting cells, wherein the antigen presenting cells are then exposed to the therapeutic.

2. The method of claim 1, wherein the therapeutic is an antigen of interest, an activating compound, or a therapeutic compound.

3. The method of claim 2, wherein the antigen of interest is a cancer antigen.

4. The method of any one of claim 3, wherein the disease or condition is cancer.

5. The method of claim 1, wherein the antigen presenting cells are macrophages, dendritic cells or B cells.

6. The method of claim 1, wherein the antigen presenting cells comprise at least one C3 receptor.

7. The method of claim 1, wherein the nanoparticle is a liposome.

8. The method of claim 1, further comprising administering a second therapeutic to the subject.

9. A method of treating a disease or condition in a subject comprising
coating a nanoparticle with activated C3 forming a nanoparticle-C3 conjugate,
administering to a subject a nanoparticle-C3 conjugate, wherein the nanoparticle-C3 conjugate comprises a therapeutic, wherein the nanoparticle-C3 conjugate targets antigen presenting cells, wherein the antigen presenting cells are then exposed to the therapeutic.

10. The method of claim 9, further comprising isolating the activated C3 from the subject prior to coating the nanoparticle.

11. A method of delivering a therapeutic to antigen presenting cells comprising administering to a subject a nanoparticle comprising a therapeutic, wherein upon administration the nanoparticle binds to activated C3 present in the subject forming a nanoparticle-C3 conjugate, wherein the nanoparticle-C3 conjugate targets antigen presenting cells.

12. The method of claim 11, wherein the therapeutic is an antigen of interest, an activating compound, or a therapeutic compound.

13. The method of claim 11, wherein the antigen of interest is a cancer antigen.

14. The method of claim 11, wherein the antigen presenting cells are macrophages, dendritic cells or B cells.

15. The method of claim 11, wherein the antigen presenting cells comprise at least one C3 receptor.

16. The method of claim 11, further comprising administering a second therapeutic to the subject.

17. The method of claim 11, wherein the nanoparticle is a liposome.

18. A method of delivering a therapeutic to antigen presenting cells comprising
coating a nanoparticle with activated C3 forming a nanoparticle-C3 conjugate,
administering to a subject a nanoparticle-C3 conjugate, wherein the nanoparticle-C3 conjugate comprises a therapeutic, wherein upon administration the nanoparticle binds to activated C3 present in the subject forming a nanoparticle-C3 conjugate, wherein the nanoparticle-C3 conjugate targets antigen presenting cells.

19. The method of claim 18, further comprising isolating the activated C3 from the subject prior to coating the nanoparticle.

20. A method of reducing tumor growth in a subject comprising administering to a subject a nanoparticle, wherein the nanoparticle comprises a tumor antigen, wherein upon administration the nanoparticle binds to activated C3 present in the subject forming a nanoparticle-C3 conjugate, wherein the nanoparticle-C3 conjugate targets antigen presenting cells, wherein the antigen presenting cells present the tumor antigen to T cells.
